(19) United States
(12) Patent Application Publication
(10) Pub. No.: US 2012/0251495 A1
(43) Pub. Date: Oct. 4, 2012

(54) PTERIN BASED THERAPIES FOR INFLAMMATORY CONDITIONS

(76) Inventors: Phillip Moheno, La Jolla, CA (US); Wolfgang Pfleiderer, Konstanz (DE); Antonio DiPasquale, San Bruno, CA (US); Arnold Rheingold, La Jolla, CA (US); Dietmar Fuchs, Innsbruck (AT)

(21) Appl. No.: 13/384,583

(22) PCT Filed: Jan. 16, 2009
(86) PCT No.: PCT/US09/31346

§ 371 Date: Jun. 18, 2012

Related U.S. Application Data
(60) Provisional application No. 61/022,156, filed on Jan. 18, 2008.

(51) Int. Cl. A61K 31/519; A61K 39/395; A61K 38/21; A61K 31/675; A61K 31/522; A61K 31/7072; A61P 35/00; A61P 35/02; A61P 37/02; A61P 29/00; A61P 31/00; A61P 25/28; A61P 25/00; A61P 3/04; A61P 9/00; A61P 3/10; A61P 9/10; A61P 25/16; A61P 19/02; A61P 17/06; A61P 19/04; A61P 11/00; A61P 11/06; A61P 13/12; A61P 1/00; A61P 37/06; A61P 1/04; A61N 5/10; A61K 35/12

(52) U.S. Cl. 424/85.7; 514/249; 424/93.7; 424/141.1; 424/278.1; 514/81; 514/50; 600/1

(57) ABSTRACT

Disclosed herein are methods for the treatment of cancer and inflammatory-based diseases and disorders, such as hepatitis B virus infection based upon the administration of calcium pterin. In one embodiment is a method of treating cancer comprising administration of dipterinyl calcium pentahydrate (DCP). In another embodiment is a method of treating hepatitis B virus infection comprising administration of dipterinyl calcium pentahydrate (DCP).

### CROSS REFERENCE

This application claims the benefit of U.S. Provisional Application No. 61/022,156, filed on Jan. 18, 2008, which is incorporated herein by reference in its entirety.

### SUMMARY OF THE INVENTION

In vivo studies of the effectiveness of various forms of calcium pterin reveal significant antitumor activity associated with (1:4 mol:mol) calcium pterin [CaPterin], (1:2 mol:mol) calcium pterin, dipterinyl calcium pentahydrate (DCP), as well as unexpectedly for a calcium chloride dihydrate solution in nude mice with MDA-MB-231 xenographs. Stepwise regression analysis of nine plasma cytokine and indoleamine 2,3-dioxygenase (IDO) metabolite levels identified four effects correlated to (1:4 mol:mol) calcium pterin administration: 1) decreased IL-6, 2) increased IL-10, 3) decreased IFN-γ, and 4) increased kynurenine. Conclusion: (1:4 mol:mol) calcium pterin [CaPterin] exerts significant (by Spearman rank order correlation) dose-response antitumor activity in nude mice with MDA-MB-231 xenographs, and sustains both inflammatory and anti-inflammatory changes in the levels of certain plasma factors.

Analysis of the cytokine changes in nude mice with MDA-MB-231 xenograph tumors resulting from the oral dosing of the antitumor agent (1:4 mol:mol) calcium pterin (CaPterin) determined that this form of calcium pterin increased plasma IL-10, decreased plasma IL-6, and decreased plasma IFN-γ (Moheno et al. in press). A plasma cytokine analysis of similarly xenographed nude mice from the previously described 2nd experiment (Moheno et al. in press) was carried out. The cytokines measured included: IL-1β, IL-2, IL-4, IL-5, IL-10, IL-12, IFN-γ, TNF-α, IL-6, and TGP-β1. The major findings from the analyses of this data are that DCP induces a significant quadratic antitumor response correlated to the derived DCP antitumor plasma cytokine pattern (DCP/APCP) which is optimized in the DCP dose range of 40-46 mg/(kg day). The DCP/APCP shows that IL-12 increases, IL-6 decreases, and IL-4 increases with decreasing relative tumor volume in the context of DCP dosing. The finding that decreased plasma IL-6 correlates with DCP antitumor efficacy is in concordance with the previous findings on the plasma cytokine effects of CaPterin. DCP is emerging as a promising new cytokine-mediated antitumor agent.

DCP also has utility as a therapy for the treatment of hepatitis B infection. DCP induces a significant dose-response reduction of Log liver HBV DNA (PCR) in female HBV mice. DCP also increased HBe antigen (ELISA) among male mice. However, DCP did not affect the serum concentrations of the IDO metabolites, tryptophan (Trp) and kynurenine (Kyn), and the Kyn/Trp ratio, except for tryptophan (Trp) at 23.0 mg/(kg day) among male HBV mice. Nevertheless, these three IDO-related measures were broadly elevated in female mice compared to male mice. The serum concentration of the chemokine RANTES was decreased in male HBV mice by 2.3 mg/(kg day) DCP. Serum cytokines, IL-4, IL-9, and IL-12, were elevated by 7.3 mg/(kg day) DCP among females.

Immunomodulation via IDO or TDO (tryptophan 2,3-dioxygenase) pathways are proposed to be involved in the modulation of HBV expression in the transgenic mice and in the anti-HBV mechanism of DCP, based upon DCP's gender-specific inhibition of viral replication, and the correlation of elevated IDO metabolites with reduced viral parameters in female HBV mice independent of DCP-treatment.

In one embodiment is a method of treating cancer comprising administration of a composition comprising calcium pterin. In another embodiment is the method wherein the calcium pterin has a stoichiometry of 1:4/calcium:pterin. In another embodiment is the method wherein the calcium pterin has a stoichiometry of 1:2/calcium:pterin. In another embodiment is the method wherein the calcium pterin is dipterinyl calcium pentahydrate (DCP).

In another embodiment is the method wherein administration of the composition results in decreased IL-6 levels. In another embodiment is the method wherein administration of the composition results in increased IL-10 levels. In another embodiment is the method wherein administration of the composition results in decreased IFN-γ levels. In another embodiment is the method wherein administration of the composition results in increased kynurenine levels. In another embodiment is the method wherein administration of the composition results in increased IL-12 levels. In another embodiment is the method wherein administration of the composition results in decreased IL-6 levels. In another embodiment is the method wherein administration of the composition results in increased IL-4 levels. In another embodiment is the method wherein administration of the composition results in inhibition of indoleamine 2,3-dioxygenase.

In another embodiment is the method wherein administering the composition is through oral, parenteral, intravenous, subcutaneous, intrathecal, intramuscular, buccal, intranasal, epidural, sublingual, pulmonary, local, rectal, or transdermal administration.

In another embodiment is the method further comprising additional therapies selected from one or more of radiation therapy, chemotherapy, high dose chemotherapy with stem cell transplant, hormone therapy, and monoclonal antibody therapy.

In another embodiment is the method wherein the cancer is selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

In another embodiment is a method of modulating the immune response comprising administration of a composition comprising calcium pterin.

In another embodiment is a method of treating an inflammatory-based disease or disorder comprising administration of a composition comprising calcium pterin. In another embodiment is the method wherein the inflammatory-based disease or disorder is selected from infectious diseases, neurodegenerative disorders, multiple sclerosis, HIV-associate dementia, AIDS dementia, Alzheimer's disease, central nervous system inflammation, obesity, dementia (various forms), coronary heart disease, diabetes (Type 1 and Type 2), atherosclerosis, chronic inflammatory diseases, autism, neonatal onset multisystem inflammatory disease, (also known as NOMID, Chronic Neurologic Cutaneous and Articular Syndrome, or CINCA), Parkinson's Disease, rheumatoid arthritis, osteoarthritis, tendinitis, bursitis, inflammatory lung disease, psoriasis, chronic obstructive pulmonary disease, lupus erythematosus, organ inflammation (eg. myocarditis, asthma, nephritis, colitis), inflammatory bowel disease (IBD), autoimmune disease, inflammatory bowel syndrome (IBS), Crohn's Disease, Chronic Ulcerative Colitis, transplant rejection, sepsis, disseminated intravascular coagulation (DIC), septic shock, psoriasis, emphysema and ischemia-reperfusion injury. In another embodiment is the method wherein administering the composition is through oral, parenteral, intravenous, subcutaneous, intrathecal, intramuscular, buccal, intranasal, epidural, sublingual, pulmonary, local, rectal, or transdermal administration.

In another embodiment is the method wherein the inflammatory-based disease or disorder is hepatitis B virus infection. In another embodiment is the method wherein administering the composition is through oral, parenteral, intravenous, subcutaneous, intrathecal, intramuscular, buccal, intranasal, epidural, sublingual, pulmonary, local, rectal, or transdermal administration. In another embodiment is the method further comprising additional therapies selected from one or more of interferon α, pegylated intereron α-2a, lamivudine, adefovir, tenofovir, telbivudine and entecavir. In another embodiment is the method wherein the calcium pterin is dipterinyl calcium pentahydrate (DCP).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF TILE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: Fourier Transformed Infrared Spectra of A) pterin, B) (1:4 mol:mol) calcium pterin, and C) dipterinyl calcium pentahydrate (DCP) (SD045).

FIG. 2: X-ray crystallographic structure of A) calcium pterin, and B) dipterinyl calcium pentahydrate (DCP).

FIG. 3: (1st experiment) Twenty-three athymic nude (nu/nu) female mice, ages 3-4 weeks, were inoculated with 5×106 MDA-MB-231 cancer cells subcutaneously into the right flank of each mouse. When tumors reached 3-5 mm in size, twenty of the mice were divided into four treatment groups of five each. Two mice with non-tumor takes were subsequently excluded. Experimental groups were treated by oral gavage once daily with the indicated test suspensions. The control group was treated with sterile water. Overall ANOVA p=0.0175; t p<0.0001 versus Control.

FIG. 4: (2nd experiment) Twenty-nine athymic nude (nu/nu) female mice, ages 3-4 weeks, were inoculated with 10×106 MDA-MB-231 cancer cells subcutaneously into the right flank of each mouse. When tumors reached 3-5 mm in size, twenty-five of the mice were divided into five treatment groups of five mice each. Four mice were assigned as controls. Four mice with non-tumor takes were subsequently excluded. Experimental groups were treated by oral gavage once daily with the indicated test suspensions or solution. The control group was untreated. Overall ANOVA p<0.0001; t p<0.0001 versus Control.

FIG. 5: Dose-response plot of Day 60 Relative Tumor Volume versus (1:4 mol:mol) calcium pterin (CaPterin) for mice in the 1st experiment (n=14) with Spearman rank order correlation, rs=0.70; p=0.005.

FIG. 6: Day 60 Relative Tumor Volumes plotted versus ACIP scores (Antitumor Cytokine/IDO Pattern).

FIG. 7: The calcium equivalent dosed to each group of mice from the 1st and 2nd experiments is plotted along with the relative tumor volumes recorded at days 42 or 43 of treatment.

FIG. 8: shows cytokine as function of Ca-Pterin dose.

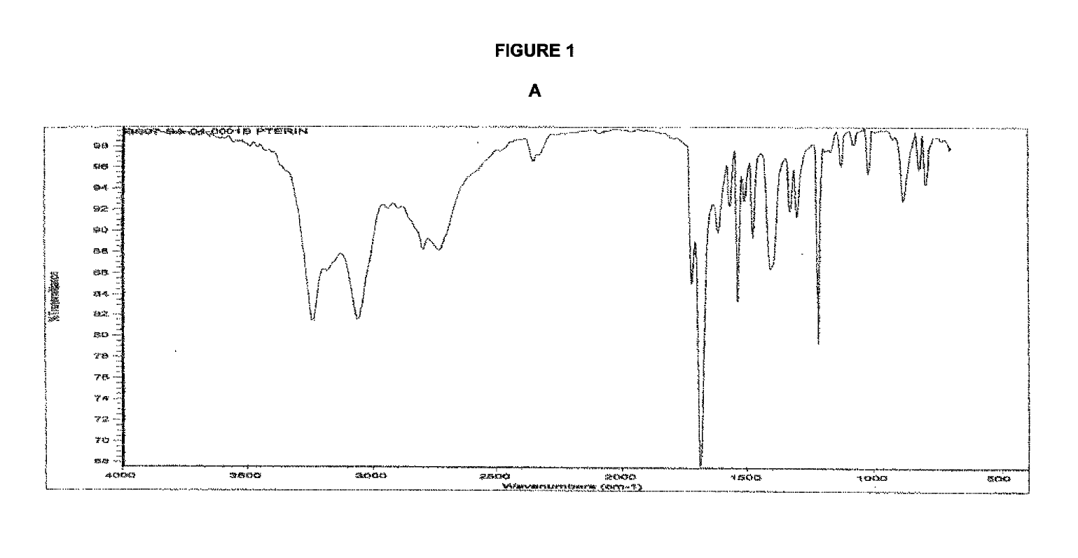

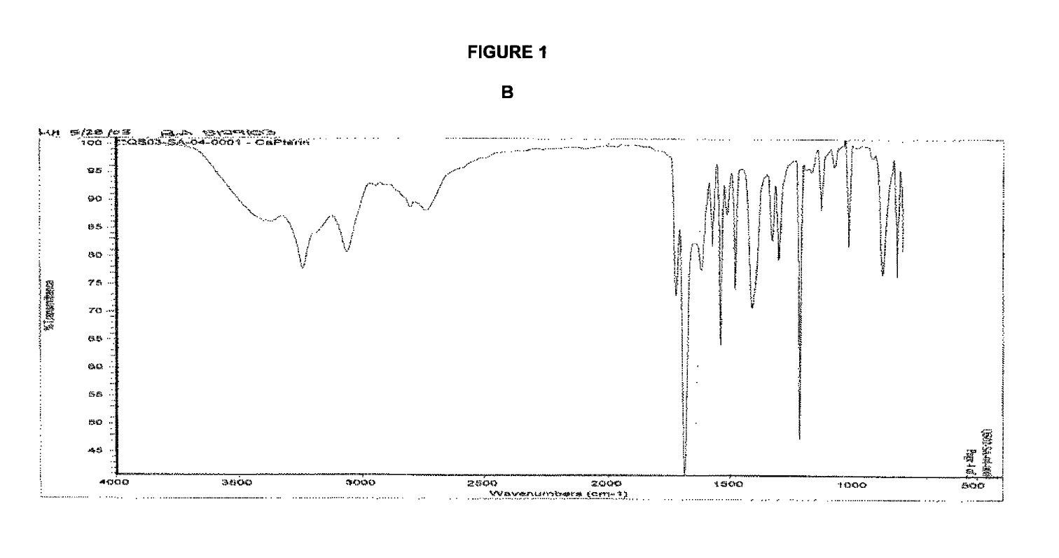

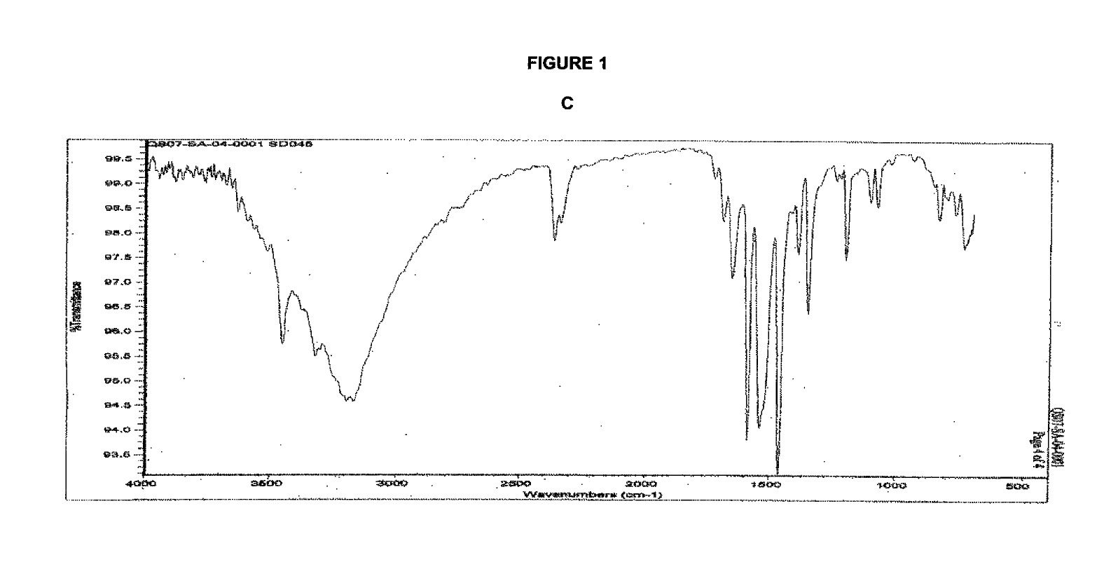

FIG. 9: Combined relative tumor growth data from 1st and 2nd experiments (Moheno et al. in press) comparing dosed calcium equivalents to Day 42,43 relative tumor volumes by treatment. The 42,43-day time points represent the longest time period in which all the FIG. 1 mice survived without confounding reactions. The dosed calcium equivalent for each treatment is given as reference. The ungavaged controls from the 2nd experiment were excluded from this analysis. Overall ANOVA for Relative Tumor Volume: p=0.002.
* p<0.05 vs. Control by Donnett T3 Post Hoc Test

FIG. 10: Combined plasma IL-12 data from 1st and 2nd experiments (Moheno et al. in press) comparing dosed calcium equivalents to plasma IL-12 levels by treatment at sacrifice. The dosed calcium equivalent for each treatment is given as reference. The ungavaged controls from the 2nd experiment were excluded from this analysis. Overall ANOVA for Plasma IL-12: p=0.001.
* p<0.05 vs. Control by Donnett T3 Post Hoc Test

FIG. 11: Relative tumor volumes and DCP/APCP levels at Day 42,43 in response to dosing for the DCP+ control mice. Control (n=3; from 1st experiment); 23 mg/(kg d) DCP (n=5; from 2nd experiment); 69 mg/(kg d) DCP (n=4; from 2nd experiment).

FIG. 12: Relative tumor volumes and IL-12 levels for the DCP+ control mice.

FIG. 13: IL-12 levels in response to DCP dosing for the DCP+ control mice.

FIG. 14: Relative tumor volume versus DCP antitumor plasma cytokine pattern for DCP+ control treated mice from FIG. 11.

FIG. 15: Mean Log [relative liver HBV DNA] values, as measured by quantitative PCR, is graphed by treatment group and gender for HBV mice (ADV=Adefovir dipivoxil). * (p<0.05)—Mean Log [relative liver HBV DNA (PCR)] is significantly different than controls of the same gender. ^{####} (p≦001)—Mean Log [relative liver HBV DNA (PCR)] is significantly different by gender.

FIG. 16: Mean HBe antigen, as measured by ELISA (PEI units/ml), is graphed by treatment group and gender for HBV mice (ADV=Adefovir dipivoxil). * (p<0.05)—Mean HBe ELISA is significantly different than controls of the same gender. ^{#} (p<0.05); ^{##} (p<0.01); ^{###} (p<0.005)—Mean HBe ELISA is significantly different by gender.

FIG. 17: Mean serum RANTES is graphed by treatment group and gender for HBV mice. * (p<0.05)—Mean serum RANTES concentration is significantly different than controls of the same gender. ^{#} (p<0.05)—Mean serum RANTES concentration is significantly different by gender.

FIG. 18: Tryptophan, kynurenine, and their ratio (Kyn/Trp) in HBV transgenic mice treat with ADV, DCP or placebo. A) mean serum tryptophan (uM), B) mean serum kynurenine (uM), and C) mean serum Kyn/Trp (uM/mM). ^{#} (p<0.05); ^{###} (p<0.005); ^{####} (p≦001) compared between genders; *** (p<0.005) compared to placebo treatment.

FIG. 19: The linear dose-response plot of Log [Liver HBV DNA (PCR)] versus DCP dosage, with significance values, is given for females (N=40), males (N=39), and all HBV mice (N=79). n.s.=non significant. The significant regression equation for the females is: Log [Liver HBV DNA (PCR)]=1.59−0.033 DCP.

FIG. 20: Standardized partial regression plots for the stepwise regression of plasma IL-6, IL-1β, IL-2, IL-4, IL-10, IL-12, and IFN-γ measures from the Table 1 data versus Day 42,43 relative tumor volume As detailed in Table 8.

### DETAILED DESCRIPTION OF THE INVENTION

This invention relates to novel pterin analogs to dipterinyl calcium pentahydrate (DCP) which possesses potent antineoplastic activity and potent anti-hepatitis B activity.

The present invention is directed to novel metal pterin and pterin analog complexes of the formula

(MXₐ)(Pterins)₂ (I)

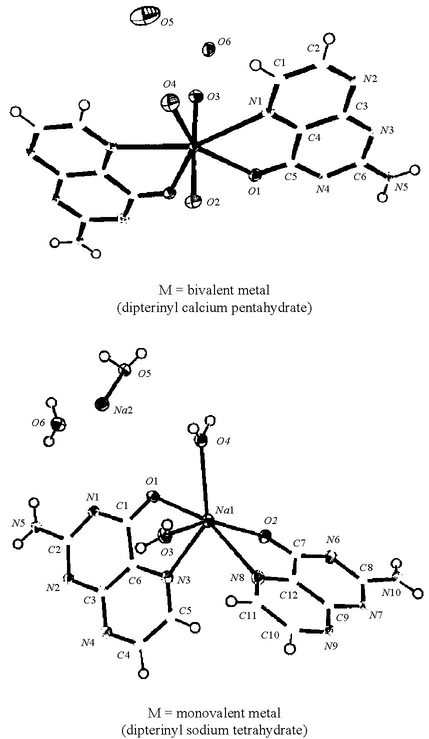

wherein

M is a monovalent or bivalent metal ion selected from the group consisting of Li1+, Na1+, K1+, Rb1+, Cs1+, Fr1+, Cu1+, Ag1+, Au1+, Hg1+, Tl1+, Cl1+, Br1+, I1+, At1+, Ca2+, Cu2+, Mg2+, V2+, Cr2+, Mn2+, Fe2+, Co2+, Zn2+, Mo2+, Sr2+, Ba2+, Ra2+, Ru2+, Rh2+, Pd2+, Cd2+, Sn2+, W2+, Re2+, Os2+, Ir2+, Pt2+, Si2+, and Sm2+;

X is an anion of an acid and has a charge of −1 or −2 when ionized;

a is an integer of from 1 to 2;

### 1. DEFINITIONS

“Pterins” refers to the following compounds which can exist as the tautomers

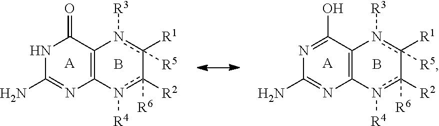

wherein

R1 and R2 are independently selected from the group consisting of hydrogen, alkyl, perhaloalkyl, carboxyl, amido, carboxamido, oxo, carboxy esters, amino, halogen, haloalkyl, hydroxy, alkoxy, azido, acylalkyl, hydroxyallyl, —C(O)H, aryl, alicyclic, aralkyl, thioalkyl, sulfhydryl (—SH), sulfonyl (SO2-3), —CN, perhaloalkoxy, and acyl;

R5 and R6 are independently selected from the group consisting of hydrogen, alkyl, perhaloalkyl, carboxyl, amido, carboxamido, oxo, carboxy esters, amino, halogen, haloalkyl, hydroxy, alkoxy, azido, acylalkyl, hydroxyalkyl, —C(O)H, aryl, alicyclic, aralkyl, thioallyl, sulfhydryl (—SH), sulfonyl (SO2-3), —CN, perhaloalkoxy, acyl, and null;

R3 and R4 are independently selected from the group consisting of —H, alkyl, —C(O)H, acyl, hydroxyalkyl, aryl, alkylaryl, hydroxy, oxo, acylalkyl, haloalkyl, perhaloalkyl, haloaryl, carboxyl, and null.

The dotted lines in the above structures represent optional bonds. The nitrogens in the B-ring can be neutral or positively charged. Thus, “Pterins” refers to both Pterin and pterin analogs including, but not limited to pterin, xanthopterin, and isoxanthopterin.

“Suspension” refers to the state of a substance when its particles are mixed but undissolved in a fluid or solid.

“RCOOH” refers to carboxylic acids, where R is alkyl, aryl, or aralkyl. Suitable anion carboxylic acids include CH3COO—, and phenyl-COO—.

“Alkyl” refers to saturated and unsaturated aliphatic groups including straight-chain, branched chain, and cyclic groups. Alkyl groups may be optionally substituted. Alkyl groups may contain double or triple bonds. Suitable alkyl groups include methyl.

“Aryl” refers to aromatic groups which have 5-14 ring atoms and at least one ring having a conjugated pi electron system and includes carbocyclic aryl, heterocyclic aryl, and biaryl groups, which may be optionally substituted. Suitable aryl groups include phenyl.

Carbocyclic aryl groups are groups wherein the ring atoms on the aromatic ring are carbon atoms. Carbocyclic aryl groups include monocycle and carbocyclic aryl groups and polycyclic or fused compounds such as optionally substituted naphthyl groups.

Heterocyclic aryl or heteroaryl groups are groups having from 1 to 4 heteroatoms as ring atoms in the aromatic ring and the remainder of the ring atoms being carbon atoms. Suitable heteroatoms include oxygen, sulfur, and nitrogen. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The term “biaryl” represents aryl groups containing more than one aromatic ring including both fused ring systems and aryl groups substituted with other aryl groups. Such groups may be optionally substituted. Suitable biaryl groups include naphthyl and biphenyl.

The term “alicyclic” means compounds which combine the properties of aliphatic and cyclic compounds. Such cyclic compounds include but are not limited to, aromatic, cycloalkyl and bridged cycloalkyl compounds. The cyclic compound includes heterocycles. Cyclohexenylethyl and cyclohexylethyl are suitable alicyclic groups. Such groups may be optionally substituted.

The term “optionally substituted” or “substituted” includes groups substituted by one to four substituents, independently selected from lower alkyl, lower aryl, lower aralkyl, lower alicyclic, hydroxy, lower alkoxy, lower aryloxy, perhaloalkoxy, aralkoxy, halo, azido, amino, acyl, lower alkylthio, oxo, acylalkyl, carboxy esters, carboxyl, carboxamido, nitro, acyloxy, alkylaryl, alkoxyaryl, phosphono, sulfonyl, hydroxyalkyl, haloalkyl, cyano, lower alkoxyalkyl, lower perhaloalkyl, and aralkyloxyalkyl.

The term “aralkyl” refers to an alkyl group substituted with an aryl group. Suitable aralkyl groups include benzyl, picolyl, and the like, and may be optionally substituted. The term “-aralkyl-” refers to a divalent group -aryl-alkylene-.

The term “lower” referred to herein in connection with organic radicals or compounds respectively defines such as with up to and including 10, preferably up to and including 6, and advantageously one to four carbon atoms. Such groups may be straight chain, branched, or cyclic.

The term “acyl” refers to —C(O)R where R is H, alkyl, and aryl.

The term “carboxy esters” refers to —C(O)OR where R is alkyl, aryl, aralkyl, and alicyclic, all optionally substituted.

The term “carboxyl” refers to —C(O)OH.

The term “oxo” refers to =0 in an alkyl group.

The term “amino” refers to —NRR′ where R and R′ are independently selected from hydrogen, alkyl, aryl, aralkyl and alicyclic, all except H are optionally substituted; and R and R1 can form a cyclic ring system.

The term “halogen” or “halo” refers to —F, —Cl, —Br and —I.

The term “cyclic alkyl” or “cycloalkyl” refers to alkyl groups that are cyclic. Suitable cyclic groups include norbornyl and cyclopropyl. Such groups may be substituted.

The term “heterocyclic” and “heterocyclic alkyl” refer to cyclic groups containing at least one heteroatom. Suitable heteroatoms include oxygen, sulfur, and nitrogen. Heterocyclic groups may be attached through a nitrogen or through a carbon atom in the ring. Suitable heterocyclic groups include pyrrolidinyl, morpholino, morpholinoethyl, and pyridyl.

The term “phosphono” refers to —PO3R2, where R is selected from the group consisting of —H, alkyl, aryl, aralkyl, and alicyclic.

The term “sulphonyl” or “sulfonyl” refers to —SO3R, where R is H, alkyl, aryl, aralkyl, and alicyclic.

The term “alkylene” idea to a divalent straight chain, branched chain or cyclic saturated aliphatic group.

The term “aralkyloxyalkyl-” refers to the group aryl-alk-O-alk- wherein “alk” is an alkylene group. “Lower aralkyloxyalkyl-” refers to such groups where the alkylene groups are lower alkylene.

The term “-alkoxy-” or “-alkyloxy-” refers to the group -alk-O— wherein “alk” is an alkylene group.

The term “alkoxy-” refers to the group alkyl-O—.

The term “-alkoxyalkyl-” or “-alkyloxyalkyl-” refer to the group -alk-O-alk- wherein each “alk” is an independently selected alkylene group. In “lower -alkoxyalkyl-”, each alkylene is lower alkylene.

The terms “alkylthio-” and “-alkylthio-” refer to the groups alkyl-S—, and -alk-S—, respectively, wherein “alk” is alkylene group.

The term “-alkylthioalkyl-” refers to the group -alk-S-alk- wherein each “alk” is an independently selected alkylene group. In “lower -alkylthioalkyl-” each alkylene is lower alkylene.

The terms “amido” or “carboxamido” refer to NR2-C(O)— and RC(O)—NR1-, where R and R1 include H, alkyl, aryl, aralkyl, and alicyclic.

The term “perhalo” refers to groups wherein every C—H bond has been replaced with a C-halo bond on an aliphatic or aryl group. Suitable perhaloalkyl groups include —CF3 and —CFCl2.

The term “pharmaceutically acceptable salt” includes salts of compounds of formula I and their prodrugs derived from the combination of a compound of this invention and an organic or inorganic acid or base.

The term “prodrug” as used herein refers to any compound that when administered to a biological system generates the “drug” substance either as a result of spontaneous chemical reaction(s) or by enzyme catalyzed or metabolic reaction(s). Prodrugs are formed using groups attached to functionality, e.g. HO—, HS—, HOOC—, R2N—, associated with the Pterins, that cleave in vivo. Prodrugs include but are not limited to carboxylate esters where the group is alkyl, aryl, aralkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. The groups illustrated are exemplary, not exhaustive, and one skilled in the art could prepare other known varieties of prodrugs. Such prodrugs of the compounds of formula I, fall within the scope of the present invention.

Inflammatory Conditions Treatable with DCP

Given that DCP has anti-inflammatory properties as evidenced in particular by its ability to 1) decrease IL-6, 2) increase IL-10, and 3) decrease IFN-γ, in nude mice with MDA-MB-231 xenographs, the following list of inflammatory-based indications can be advantageously treated by DCP or one or more of its analogs described above.
infectious diseases
neurodegenerative disorders
multiple sclerosis
HIV-associate dementia
AIDS dementia
Alzheimer's disease
central nervous system inflammation
obesity
dementia (various forms)
coronary heart disease
diabetes (Type 1 and Type 2)
atherosclerosis
chronic inflammatory diseases
autism
neonatal onset multisystem inflammatory disease
(also known as NOMID, Chronic Neurologic Cutaneous and Articular Syndrome, or CINCA)
Parkinson's Disease
rheumatoid arthritis
osteoarthritis
tendinitis
bursitis
inflammatory lung disease
psoriasis
chronic obstructive pulmonary disease
lupus erythematosus
organ inflammation (eg. myocarditis, asthma, nephritis, colitis)
inflammatory bowel disease (IBD)
autoimmune disease
inflammatory bowel syndrome (IBS)
Crohn's Disease
Chronic Ulcerative Colitis
transplant rejection
sepsis
disseminated intravascular coagulation (DIC)
septic shock
psoriasis
emphysema
ischemia-reperfusion injury

Since the discovery and elucidation of the anti-tumor properties of calcium pterin (Moheno, 2004; Winkler et al., 2006), it has become important to identify stable, effective calcium pterin complex forms, as well as to further specify their immuno-mechanism(s) of action. The current study reports on advances in both these areas, and the synthesis and characterization of a promising new cancer therapeutic, dipterinyl calcium pentahydrate (DCP).

A suspension of calcium pterin in the molar ratio of 1:4 calcium to pterin (2-amino-4(3H)-pteridinone) known as CaPterin was found to possess significant antitumor efficacy against MDA-MB-231 human breast xenographs in nude mice, as well as highly significant activity against spontaneous mammary gland tumors in C3H/HeN-MTV+ mice, based upon National Cancer Institute standards (Moheno, 2004). An immunomodulatory mode of action for CaPterin was deduced by comparing the antitumor efficacy of CaPterin in four different mouse/tumor systems: i.e., the two cited above, as well as in Balb/c mice with EMT6 xenographs and SCID mice with MDA-MB-231 xenographs. The further specification of the immunological effects of CaPterin in nude mice with MDA-MB-231 xenographs is herein described. In the present study, the nude mouse tumor system was chosen because of the human origin of the tumor xenographs and the uniformity of the tumors produced. In an effort to expand the characterization of the active forms of calcium pterin, antitumor data are also presented for (1:2 mol:mol) calcium pterin, as well as for dipterinyl calcium pentahydrate at two dosages. In addition, comparative antitumor efficacy results are given for pterin and a calcium chloride dihydrate solution, the latter at a Ca⁺² concentration equivalent to that contained in the (1:4 mol:mol) calcium pterin suspension [CaPterin] administered at 21 mg/kg/day.

Because in vitro studies suggest that the antitumoral effects of CaPterin involve the immunomodulatory actions of N K cell activation and indoleamine 2,3-dioxygenase (IDO) inhibition (Moheno et al., 2005; Winlder at al., 2006), the investigators herein analyze the in vivo immunological effects evoked by CaPterin based upon the measurement of ten plasma components: IL-1b, IL-2, IL-4, IL-6, IL-10, IL-12, IFN-γ, TNF-α, tryptophan (Trp), and kynurenine (Kyn). Kynurenine/tryptophan (Kyn/Trp) ratios were calculated as a measure of IDO activity (Wirleitner et al., 2003), previously shown to be inhibited in vitro by CaPterin in human PBMCs (peripheral blood mononuclear cells) (Winkler et al., 2006).

### 2. MATERIALS AND METHODS

### 2.1. Test Substances

2.1.1. (1:4 mol:mol) Calcium Pterin Suspension [CaPterin] (1 mg/ml):

Suspension A is prepared by mixing 24 mg pterin (Schircks Laboratories, Jona, Switzerland) into 30 ml distilled H₂O. Suspension B is prepared by first dissolving 8 mg CaCl₂.2H₂O into 10 ml distilled H₂O, then mixing in 8 mg pterin. Suspension B is then mixed with Suspension A yielding 40 ml of 1 mg/ml (1:4 mol:mol) calcium pterin suspension [CaPterin].

2.1.2. Pterin Suspension (1 mg/ml):

Prepared by mixing 40 mg pterin in 40 ml distilled H₂O.

2.1.3. (1:2 mol:mol) Calcium Pterin Suspension (1.2 mg/ml):

Suspension A is prepared by mixing 16 mg pterin into 30 ml distilled H₂O. Suspension B is prepared by first dissolving 16 mg CaCl₂.2H₂O into 10 ml distilled H₂O, then mixing in 16 mg pterin. Suspension B is then mixed with Suspension A yielding 40 ml of 1.2 mg/ml (1:2 mol:mol) calcium pterin suspension.

2.1.4. Dipterinyl Calcium Pentahydrate (DCP) Synthesis:

Pure pterin (81.7 mg, 0.5 mmol) was dissolved in H₂O (50 ml) and 0.1 N NaOH (6 ml) and CaCl₂.2H₂O (36.7 mg, 0.25 mmol) was added to the clear solution with stirring (pH 10.93). A yellowish precipitate was formed within a few minutes. Stirring was continued for 1 day and then the precipitate collected and dried in a vacuum desiccator to give 75 mg. The elemental analysis is consistent with (C₆H₄N₅O)₂Ca.5H₂O (MW 454.4).

| | | | | | | |
|---|---|---|---|---|---|---|
| Calc. | C | 31.74 | H | 4.00 | N | 30.85 |
| Found | C | 31.22 | H | 3.97 | N | 29.83 |

Comparison of the extinctions of the UV spectra of pterin and (C₆H₄N₅O)₂Ca.5H₂O taken at pH 13 give the following:

| Pterin: | 223 nm | 250 nm | 357 nm |
|---|---|---|---|
| | (8,700); | (21,380); | (8,510). |
| (C₆H₄N₅O)₂Ca•5H₂O: | 223 nm | 250 nm | 357 nm |
| | (14,450); | (39,810); | (13,490). |

2.1.5. Dipterinyl Calcium Pentahydrate (DCP) Suspensions:

A 1.1 mg/ml suspension was prepared by mixing 44 mg dipterinyl calcium pentahydrate in 40 ml distilled H₂O. A 3.3 mg/ml suspension was prepared by mixing 132 mg dipterinyl calcium pentahydrate in 40 ml distilled H₂O.

2.1.6. CaCl₂.2H2O Solution (0.2 mg/ml):

Prepared by dissolving 8 mg CaCl₂.2H₂O into 40 ml distilled H₂O.

### 2.2. Fourier Transformed Infrared Spectrophotometry

Infrared spectra (FIG. 1) were determined using a Nicolet Impact 400 QSE 335/045, by Quadrant Scientific, Inc. (San Diego, Calif.).

### 2.3. X-ray Crystallographic Analysis

2.3.1. Calcium Pterin

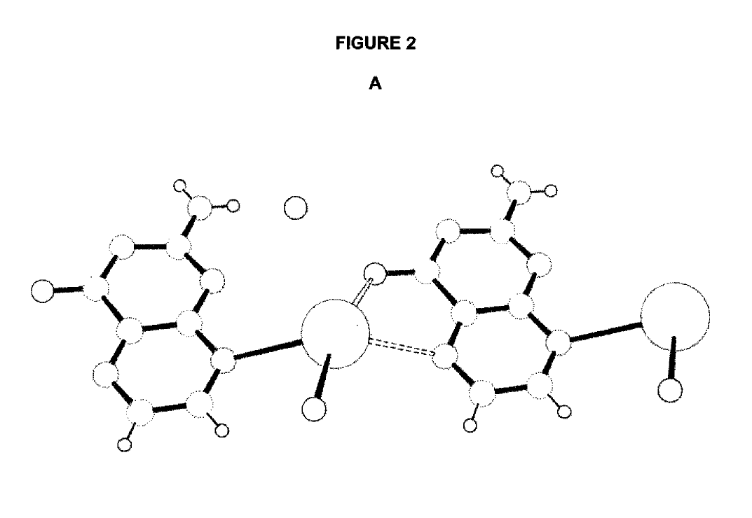

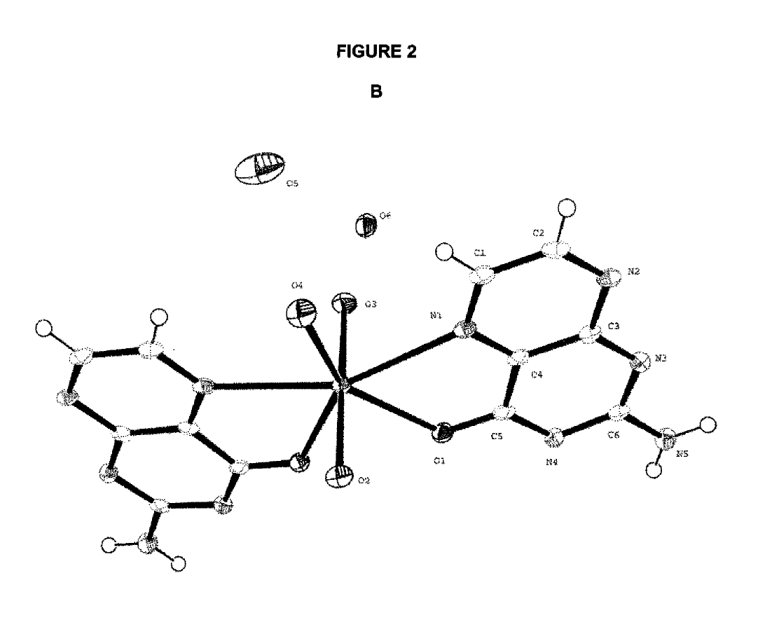

Crystal plates were grown from (1:4 mol:mol) calcium pterin suspension after solubilization with mild aqueous NaOH. A yellow plate 0.08×0.08×0.03 mm in size was mounted on a Cryoloop with Paratone oil. Data were collected in a nitrogen gas stream at 100(2) K using phi and omega scans. Crystal-to-detector distance was 60 mm and exposure time was 20 seconds per frame using a scan width of 0.5°. Data collection was 99.4% complete to 25.00° in θ. A total of 7203 reflections were collected covering the indices, −9<=h<=8, −20<=k<=19, −9<=l<=11. 1843 reflections were found to be symmetry independent, with an Rᵢₙₜ of 0.0932. Indexing and unit cell refinement indicated a primitive, monoclinic lattice. The space group was found to be P2(1)/c (No. 14). The data were integrated using the Bruker SAINT software program and scaled using the SADABS software program. Solution by direct methods (SIR-97) produced a complete heavy-atom phasing model consistent with the proposed structure. All non-hydrogen atoms were refined anisotropically by full-matrix least-squares (SHELXL-97). All hydrogen atoms were placed using a riding model. Their positions were constrained relative to their parent atom using the appropriate HFIX command in SHELXL-97. The derived structure is given in FIG. 2A.

2.3.2. Dipterinyl Calcium Pentahydrate (DCP)

Crystal plates were grown from DCP suspension after solubilization with mild aqueous NaOH. A yellow plate 0.12×0.10×0.05 mm in size was mounted on a Cryoloop with Paratone oil. Data were collected in a nitrogen gas stream at 100(2) K using phi and omega scans. Crystal-to-detector distance was 60 mm and exposure time was 20 seconds per frame using a scan width of 0.5°. Data collection was 99.6% complete to 25.00° in θ. A total of 6501 reflections were collected covering the indices, −8<=h<=8, −19<=k<=19, −9<=l<=9. 1682 reflections were found to be symmetry independent, with a Rᵢₙₜ of 0.0561. Indexing and unit cell refinement indicated a primitive, monoclinic lattice. The space group was found to be P2(1)/m (No. 11). The data were integrated using the Bruker SAINT software program and scaled using the SADABS software program. Solution by direct methods (SIR-97) produced a complete heavy-atom phasing model consistent with the proposed structure. All non-hydrogen atoms were refined anisotropically by full-matrix least-squares (SHELXL-97). All hydrogen atoms were placed using a riding model. Their positions were constrained relative to their parent atom using the appropriate HFIX command in SHELXL-97. The derived structure for DCP is given in FIG. 2B.

### 2.4. In Vivo Testing

2.4.1. Protocol

2.4.1.1. 1^{st} Experiment

The aims of the 1^{st} experiment were to determine a dose-response curve for the (1:4 mol:mol) calcium pterin suspension, to compare the antitumor activity of this suspension to pterin alone (pterin control), and to test the effect of CaPterin mega-dosing at 100 mg/kg/day. Antitumor efficacy was evaluated in nude mice with MDA-MB-231 human tumor xenographs by Perry Scientific (San Diego, Calif.). In the 1^{st} experiment, twenty-three athymic nude (nu/nu) female mice, ages 3-4 weeks, were purchased from Harlan Sprague Dawley, Inc. (Indianapolis, Ind.). 5×10⁶ MDA-MB-231 cancer cells were injected subcutaneously into the right flank of each mouse. When tumors reached 3-5 mm in size, twenty mice were divided into four treatment/control groups of five mice each. The four treatment groups were: (1:4 mol:mol) calcium pterin (7 mg/kg/day); pterin (21 mg/kg/day); (1:4 mol:mol) calcium pterin (21 mg/kg/day); and sterile water control. Two mice with outlying tumor sizes or non-tumor takes were excluded shortly after treatment began: one from the pterin group and one from the control group. Any tumor which did not persist for >14 days was considered to be outlying statistically and was therefore not included in the final metabolic analysis. Only one outlier persisted >4 days, for 14 days. Also excluded from the metabolic analysis in the 1^{st} experiment was one mouse from each of the four experimental groups mega-dosed by oral gavage with 100 mg/kg/day CaPterin for tip to 31 days to test for toxicity. All the other mice in the 1^{st} experiment persisted ≧60 days without complications.

2.4.1.2 2^{nd} Experiment

The aims of the 2^{nd} experiment were three-fold: 1) to test the antitumor effect of the increased [Ca⁺²] in the (1:2 mol:mol) calcium pterin suspension compared to the (1:4 mol:mol) calcium pterin suspension; 2) to evaluate the antitumor efficacy of DCP at two concentrations, 23 mg/kg/day and 69 mg/kg/day; and 3) to evaluate the antitumor activity of the calcium pterin to calcium chloride alone (CaCl₂ control). In the 2^{nd} experiment, twenty-nine athymic nude, also purchased from Harlan Sprague Dawley, Inc. (Indianapolis, Ind.), were each injected subcutaneously with 10×10⁶ MDA-MB-231 cancer cells into the right flank. When tumors reached 3-5 mm in size, the mice were divided into five treatment groups of five each and a control group of four mice. The five treatment groups were: (1:4 mol:mol) calcium pterin (21 mg/kg/day); (1:2 mol:mol) calcium pterin (25 mg/kg/day); DCP (23 mg/kg/day); DCP (69 mg/kg/day); and calcium chloride, dihydrate (4.2 mg/kg/day). Four of these mice with outlying tumor sizes or non-tumor takes were excluded shortly after treatment began: one each from the (1:4 mol:mol) calcium pterin group, the (1:2 mol:mol) calcium pterin group, the DCP (69 mg/kg/day) group, and one from the control group. All the other mice in the 2^{nd} experiment persisted ≧43 days without complications. Experimental groups were treated by oral gavage once daily with the indicated test suspensions or solutions.

The control mice in the 1^{st} experiment were treated with sterile water while the control mice in the 2^{nd} experiment were untreated to evaluate the effect of mouse handling and gavaging upon tumor growth. Daily dosing was for 7 days per week. Animals were restrained but not anesthetized for oral dosing. Tumors were measured twice weekly with calipers and body weights taken twice weekly on the day of tumor measurements. Blood was collected from all animals via cardiac puncture at termination (after 70 to 98 days of treatment) and processed to EDTA plasma for analysis. Tumor size measurements for the control group in the 2^{nd} experiment on days 4 and 7 were missed due to a technical oversight.

2.4.2. Cell Line Propagation and Inoculation

The MDA-MB-231 human breast tumor cell lines were supplied by SRI International (Menlo Park, Calif.) and propagated using standard in vitro cell expansion methods. Briefly, cells were grown in L-15 media from Gibco (Cat. No. 11415-064) supplemented with 2 mM L-Glutamine and 10% Fetal Bovine Serum (FBS). The cells were cultured in an incubator with 5% CO₂, 37.5° C., and 80% humidity. Cells were harvested with 0.25% (w/v) Trypsin-0.03% (w/v) EDTA solution. Cells were prepared for injection by standard methods to appropriate concentrations. Animals were temporarily restrained but not anesthetized for the inoculation of the tumor cells. Animals were subcutaneously injected with the tumor cells in a 100-200 μl volume.

2.4.3. Animal Care

The animals were housed 4 to a cage in approved micro-isolator cages. Caging bedding and related items were autoclaved prior to use. No other species were housed in the same room(s) as the experimental animals. The rooms were well ventilated (greater than 10 air changes per hour) with 100% fresh air (no air recirculation). A 12-hour light/12-hour dark photoperiod was maintained, except when room lights were turned on during the dark cycle to accommodate study procedures. Room temperature was maintained between 16-22° C. Animal room and cage cleaning was performed according to Perry Scientific SOP (Standard Operating Procedure). Animals had ad libitum access to irradiated PicoLab Rodent Diet 20 mouse chow. Autoclaved and chlorinated, municipal tap water was available ad libitum to each animal via water bottles.

Treatment of the animals was in accordance with Perry Scientific SOP, which adhered to the regulations outlined in the USDA Animal Welfare Act (9 CFR [Code of Federal Regulations], Parts 1, 2 and 3) and the conditions specified in The Guide for Care and Use of Laboratory Animals (ILAR [Institute for Laboratory Animal Research] publication, 1996, National Academy Press). The protocol was approved by Perry Scientific's Institutional Animal Care and Use Committee prior to initiation of the study. The study conduct was in general compliance with the US FDA Good Laboratory Practice Regulations currently in effect (21 CFR, Part 58).

### 2.5. Measurements

2.5.1. Tumor Growth Rates

Each animal was individually tracked for tumor growth by external caliper measurements of protruding tumor. Primary tumor sizes were measured using calipers and an approximate tumor volume calculated using the formula 1/2 (a×b²), where b was the smaller of two perpendicular diameters.

For each group, the mean and standard error of the mean (SEM) of the ratio V/Vo, Relative Tumor Volume (RTV), were plotted as a function of treatment time after inoculation. V₀ was the tumor volume at Day 0, when treatment began.

2.5.2. Plasma Cytokines, and Tryptophan and Kynurenine Levels

Cytokine levels in EDTA plasma from the mice in the 1^{st} experiment were determined by ELISA assay at Alta Analytical Laboratories (San Diego, Calif.) using a LINCOplex Kit (Linco Research). Tryptophan (Trp) and kynurenine (Kyn) concentrations were measured from EDTA plasma samples by high pressure liquid chromatography (HPLC) using 3-nitro-L-tyrosine as the internal standard (Widner et al., 1997). To estimate IDO activity, the kynurenine to tryptophan ratio (Kyn/Trp) was calculated and expressed as μmol kynurenine/mmol tryptophan (Wirleitner, 2003). The values from five mice in the 1^{st} experiment are not included in the summary statistics given in Table 1 for the following reasons. One mouse from each of the four treatment groups was used to test the effects of mega dosing, i.e., with (1:4 mol:mol) calcium pterin at 100 mg/kg/day after Day 60 of treatment, and are excluded. Also excluded was one mouse from the (1:4 mol:mol) calcium pterin (7 mg/kg/day) group which expired suddenly a few days before blood was collected. Cytokine measurements <3.2 pg/ml are reported as n.d. (not detectable) because the standard curves used in the ELISA assays were not calibrated below that level.

2.5.3. IDO Inhibition Determined In Vitro with Human PBMCs

The purpose of this in vitro study was to measure the IC₅₀ values of IDO in PBMCs for 1) calcium pterin, CaCl₂, and pterin to determine measurable synergistic effects between Ca⁺² and pterin, and 2) to compare these values with those of DCP for the assessment of relative IDO inhibitory strength. IC₅₀ (μM) values for IDO inhibition by (1:4 mol:mol) calcium pterin, DCP, CaCl₂, and pterin were determined in vitro with human PBMCs (both PHA-stimulated and unstimulated) by measuring kynurenine production as previously described (Winkler et al., 2006).

### 2.6. Statistics

Time course statistical analyses based upon repeated measures ANOVA (Analysis of Variance) models, and standard ANOVA models for group effects, were used (StatView SE+Graphics, v 1.03). Spearman rank order correlations were calculated, and a stepwise regression analysis was carried out (SPSS Graduate Pack.

### 3. RESULTS

### 3.1. Fourier Transformed Infrared Spectrophotometry

FIG. 1 gives the FT-IR spectra of A) pterin, B) (1:4 mol:mol) calcium pterin, and C) DCP (SD045). Relative to pterin, the calcium pterin shows enhanced broad peak signaling at ˜3400 cm⁻¹ (O—H stretch), consistent with hydration. The calcium pterin spectrum, however, looses the small twin peaks at ˜2360 cm⁻¹ which are present in the pterin and DCP spectra, since both these latter two compounds have protonizable ring nitrogens (N2 and N3 in FIG. 2B) under the experimental conditions. The twin peaks at ˜2360 cm⁻¹, corresponding to these protonated ring nitrogen(s), are not present in the calcium pterin spectrum since calcium complexes with N2 (see FIG. 2A) and sterically blocks N2 (and presumably N3 as well) rendering them unavailable to protonation.

With respect to DCP, we see the following spectral changes relative to pterin: an increased broad peak, with a superimposed sharper peak, in the 3200 cm⁻¹ to 3700 cm⁻¹ range (O—H & N—H stretches) attributable to hydration and alteration of the aromatic amine electronic environment. A decreased peak, at ˜1660 cm⁻¹ (C═O stretch) is consistent with calcium complexation of the oxygen. The increased peaks at 1590 cm⁻¹ and 1540 cm⁻¹ (C═N & C═C heterocycle stretches) are unique to DCP. The increased peak at 1460 cm⁻¹ (O—H bend) is consistent with the hydration of DCP.

### 3.2. X-ray Crystallographic Analysis

The X-ray crystallographic structures of calcium pterin and dipterinyl calcium pentahydrate (DCP) are given in FIG. 2.

### 3.3 Antitumor Efficacy of Calcium Pterin

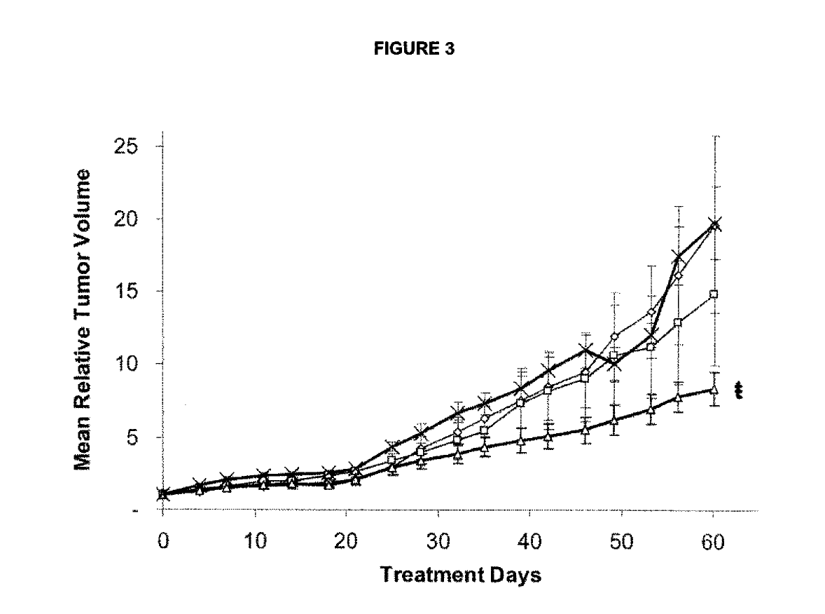

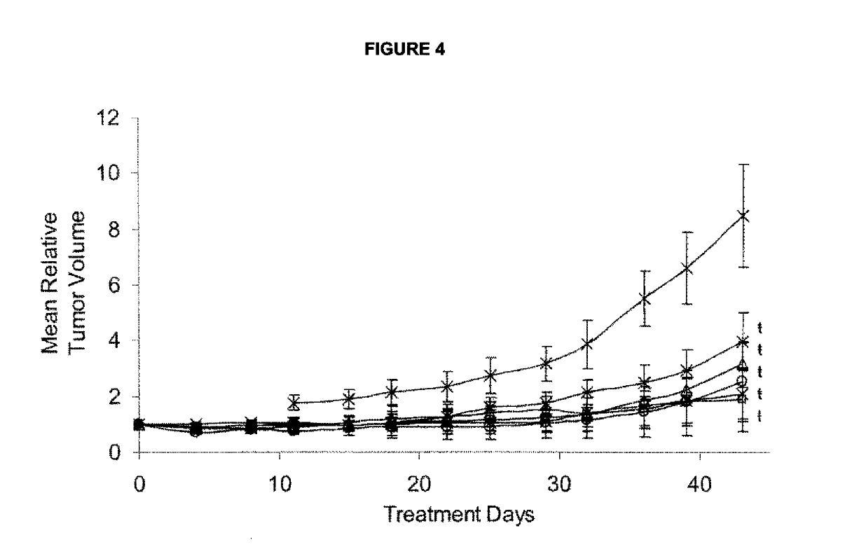

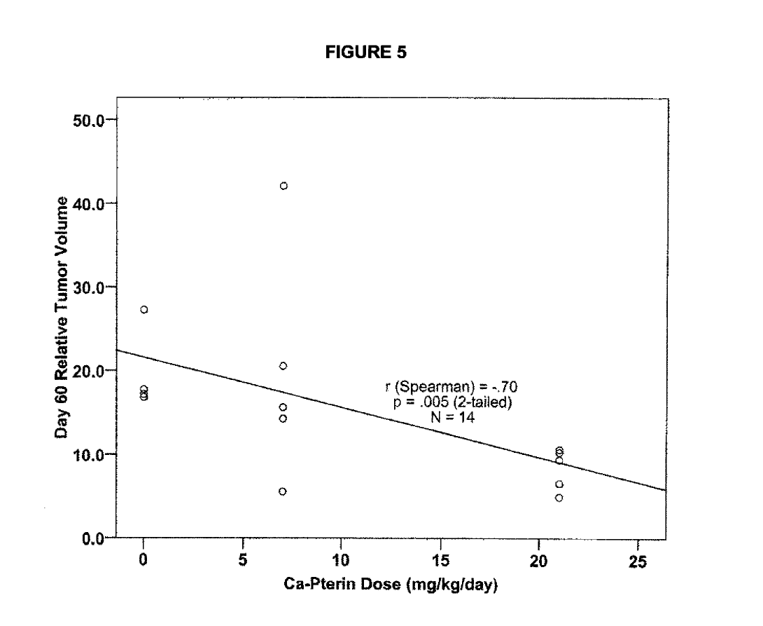

FIG. 3 (1^{st} experiment) shows that (1:4 mol:mol) calcium pterin [CaPterin] at 21 mg/kg/day significantly inhibits MDA-MB-231 human breast tumor growth in nude mice, giving a 41% T/C ratio (mean treatment tumor volume to mean control tumor volume) after 60 days in the 1^{st} experiment, and 37% T/C after 43 days in the 2^{nd} experiment (FIG. 4). The 60-day and 43-day time points represent the longest time periods in each experiment, respectively, during which all the mice survived without confounding reactions. (1:4 mol:mol) calcium pterin [CaPterin] at 7 mg/kg/day turned out to be non-significant under the conditions of the 1^{st} experiment; nevertheless, a dose-response relationship was derived (FIG. 5). Pterin at 21 mg/kg/day was tested in the 1^{st} experiment (FIG. 3) as a control and found to have no antitumor activity.

FIG. 4 (2^{nd} experiment) shows that (1:2 mol:mol) calcium pterin, dipterinyl calcium pentahydrate (DCP) at both dosages tested, and calcium chloride dihydrate significantly inhibit MDA-MB-231 xenograph growth in nude mice. These efficacy findings identify a new efficacious form of calcium pterin, dipterinyl calcium pentahydrate (DCP). Comparison of the control mice tumor growth curves from the 1^{st} experiment (gavaged with sterile water) and 2^{nd} experiment (untreated) by repeated measures ANOVA found them to be statistically indistinguishable (p=0.99).

There was no observed toxicity, as determined by body weight changes, among any of the mice in both the 1^{st} and 2^{nd} experiments. Similarly, there was no observed toxicity (appreciable weight loss) among any of the mice mega-dosed by oral gavage with 100 mg/kg/day CaPterin for up to 31 days. The greatest weight loss among the mega-dosed group was with one mouse that lost 8.1% (2.1 g) of body weight after 32 days, which included a loss of 400 mm³ of tumor mass during this period.

### 3.4. Plasma Cytokine Levels and Indoleamine 2,3-Dioxygenase (IDO) Activity

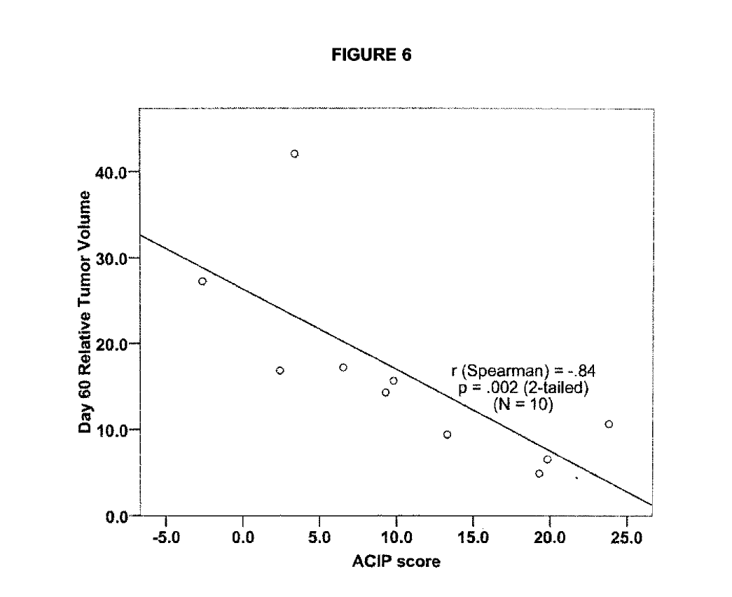

Table 1 gives the means and the SEM for the eleven plasma cytokines and IDO measures from the mice in the 1^{st} experiment, with the exclusions cited in the protocol. ANOVAs determined that none of the cytokine and IDO metabolite plasma concentrations were significantly different across treatment groups by Bonferroni criteria. The large variances for some of the group measures (e.g. IL-12: CaPterin 21 mg/kg/day) indicate that substantial variability is associated with these plasma levels. Also, no significant rank-order or linear correlations to CaPterin dosage and Day 60 Relative Tumor Volumes by these plasma measures were found. However, multivariate statistical analysis of the data derived, through stepwise regression analysis, a significant underlying pattern of cytokine and IDO metabolite effects attributable to CaPterin dosing (Table 2). For the purposes of this analysis, plasma IL-2 and IL-4 measures were excluded since they were consistently below the limits of detection (<3.2 pg/ml) and other “not detectable” values were set to 3.2 pg/ml, the lowest validated level. The other measures, including IFN-γ, had sufficient variances to be analyzable by the stepwise regression procedure. In the resultant statistically significant (p<0.047) ACIP (Antitumor Cytokine/IDO Pattern) model, plasma IL-6 and IFN-γ decrease in response to CaPterin dosage, and IL-10 and kynurenine increase. The standard regression coefficients given in Table 2 allow for direct comparison of the relative contributions from each measure in the ACIP model. The Table 2 regression was further used to calculate individual ACIP scores for each mouse which are plotted versus Day 60 Relative Tumor Volumes in FIG. 6. Partial regression plots are given in Table 2 for the four ACIP plasma measures of IL-10, IL-6, Kynurenine, and IFN-γ.

TABLE 1
Blood was collected from the mice in the 1^{st} experiment
via cardiac puncture at sacrifice after Day 60
and processed to EDTA plasma for analysis.

| | | CaPterin | CaPterin |
|---|---|---|---|
| | Control (N = 3) | (N = 3) | (N = 4) |
| | (Sterile H₂O) | (7 mg/kg/day) | (21 mg/kg/day) |
| Day 60 Rel Tum Vol | 20.4 ± 3.4 | 24.0 ± 9.0 | 7.9 ± 1.3 |
| IL-1b (pg/ml) | n.d. | 5.2 ± 2.0 | 3.4 ± 0.2 |
| IL-2 (pg/ml) | n.d. | n.d. | n.d. |
| IL-4 (pg/ml) | n.d. | n.d. | n.d. |
| IL-6 (pg/ml) | 104.5 ± 10.9 | 114.7 ± 43.2 | 121.7 ± 78.1 |
| IL10 (pg/ml) | n.d. | 41.9 ± 22.7 | 50.8 ± 32.3 |
| IL-12 (pg/ml) | n.d. | 7.7 ± 4.4 | 243.8 ± 237.8 |
| IFN-γ (pg/ml) | n.d. | 4.7 ± 0.6 | n.d. |
| TNF-α (pg/ml) | 4.3 ± 0.5 | 9.8 ± 1.5 | 8.5 ± 1.6 |
| Kynurenine (μM) | 1.4 ± 0.2 | 1.2 ± 0.2 | 1.8 ± 0.3 |
| Tryptophan (μM) | 93.6 ± 3.1 | 87.9 ± 14.6 | 106.5 ± 7.8 |
| Kyn/Trp (μM/nM) | 14.4 ± 2.1 | 14.1 ± 0.2 | 17.5 ± 3.8 |

One mouse from each of the two CaPterin groups and the Control group, i.e., three mice, were excluded from this analysis because they were used to test the effects of mega-dosing after Day 60 of treatment. Also, one mouse from the CaPterin (7 mg/kg/day) treatment group expired suddenly before cardiac puncture could be carried out. Plasma cytokine levels were determined by ELISA assay. (n.d. = not detectable; <3.2 pg/ml). IDO Metabolite levels were determined by HPLC. All values are given as Means ± SEM.

TABLE 2
The Antitumor Cytokine/IDO Pattern (ACIP) induced by
CaPterin derived from the stepwise regression of the
Table 1 data; F-to-Enter = 2.0 and F-to-Remove = 1.996.

| | | Standard |
|---|---|---|
| | Regression Model | Regression |
| | Coefficients* | Coefficients* |
| CaPterin Dose | 10.5 (Intercept) | |
| Versus: | | |
| Plasma IL-6 | −0.096 | −0.98 |
| Plasma IL-10 | 0.31 | 1.54 |
| Plasma IFN-γ | −3.16 | −0.33 |
| Plasma Kynurenine | 7.89 | 0.46 |

The model is significant to *p = .047. Standardized partial regression plots are given.

### 3.5. IDO Inhibition Determined In Vitro with Human PBMCs

Table 3 gives the IC₅₀ values for IDO inhibition determined in vitro with human PBMCs, both unstimulated and PHA-stimulated. Normal human calcium and pterin blood levels are also given for comparison. Calcium pterin (CaPterin) and DCP show significantly greater in vitro IDO inhibition than either calcium or pterin tested alone in both the unstimulated and PHA-stimulated systems.

TABLE 3
IC50 values for IDO inhibition are given, as determined
by kynurenine production in vitro for both PHA-
stimulated and unstimulated human PBMCs.

| | Unstimulated | PHA stimulated | Normal blood | |
|---|---|---|---|---|
| | IC₅₀ (MM) | IC₅₀ (MM) | plasma levels | |
| DCP | 470 | 320 | | |
| CaCl₂ | >1400 | >1400 | ~2.3 | mM * |
| pterin | >1200 | 5300 | 5-26 | nM ** |
| Ca-equiv as CaPterin | 190 | 190 | | |
| pterin-equiv as | 750 | 750 | | |
| CaPterin | | | | |

Normal human blood levels for calcium and pterin are given for comparison.
* Human plasma levels of calcium, which occurs predominantly in several complexed forms.
** Human plasma pterin levels as measured in several studies (Andondonskaja-Renz and Zeitler, 1983; Zeitler et al., 1983; Andondonskaja-Renz and Zeitler, 1984; Zeitler & Andondonskaja-Renz, 1987; Eto et al., 1992).

### 4. DISCUSSION

In the current study, (1:4 mol:mol) calcium pterin [CaPterin] at 7 mg/kg/day was found not to have significant antitumor activity in the 1^{st} experiment. In our previous study (Moheno, 2004), (1:4 mol:mol) calcium pterin [CaPterin] at 7 mg/kg/day effected a significant 41% T/C ratio. The difference in efficacies at 7 mg/kg/day reported in the two studies is attributable to the fact that the MDA-MB-231 tumors grew significantly faster in the Moheno (2004) study, i.e., 9.5-fold in 14 days, versus 2.4-fold in 14 days in the current study (FIG. 3). Faster growing tumors demonstrated a greater percent tumor growth inhibition with CaPterin. The increased tumor growth rate of the MDA-MB-231 cells in the Moheno (2004) study is likely due to the use of a faster growing clone of these cells. Other differences in experimental condition, such as the use of a different stock of nude mice or test article inconsistency, are less likely explanations.

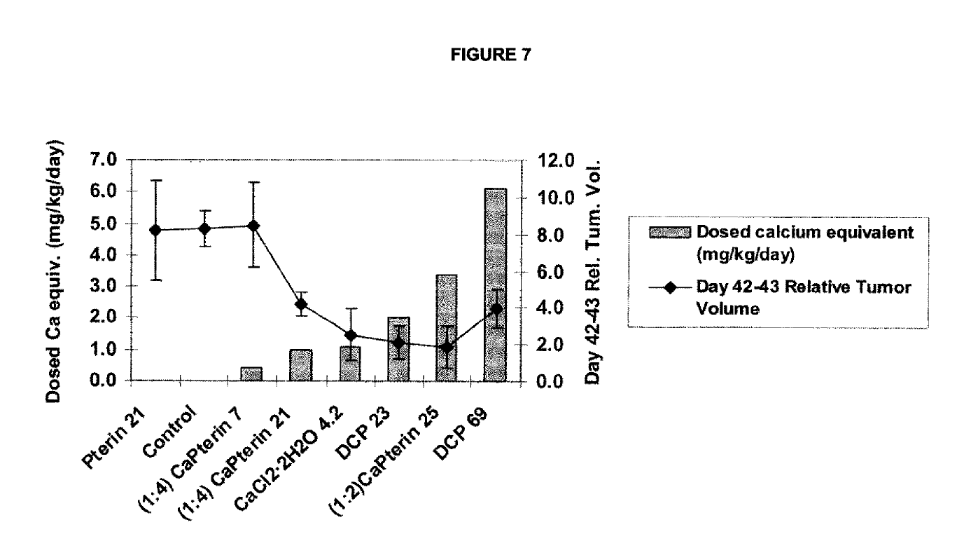

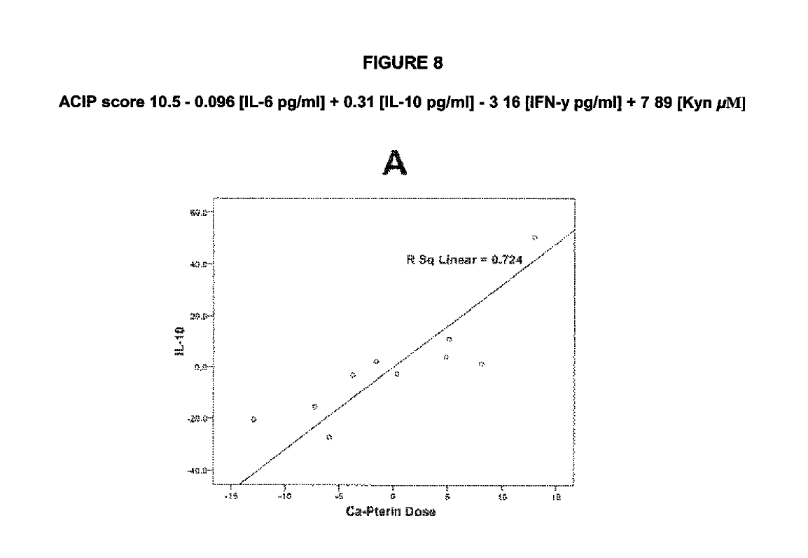

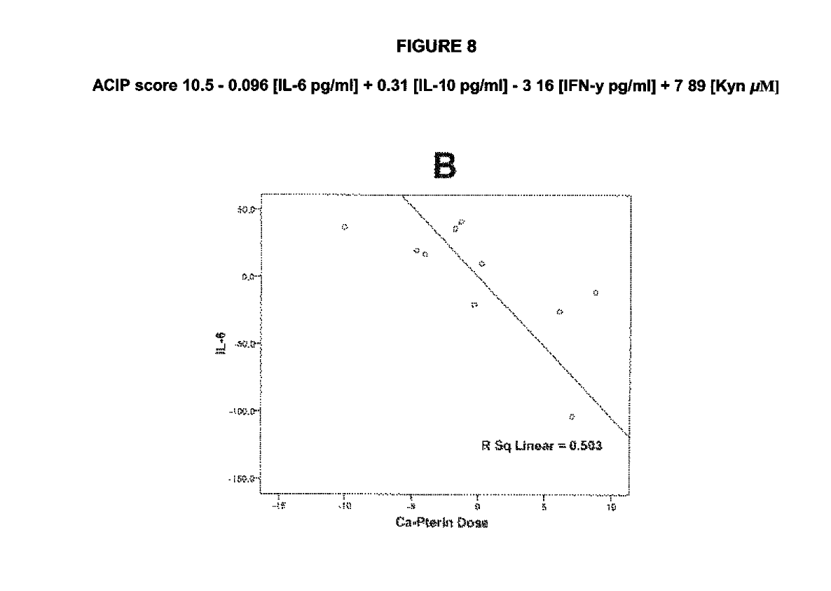

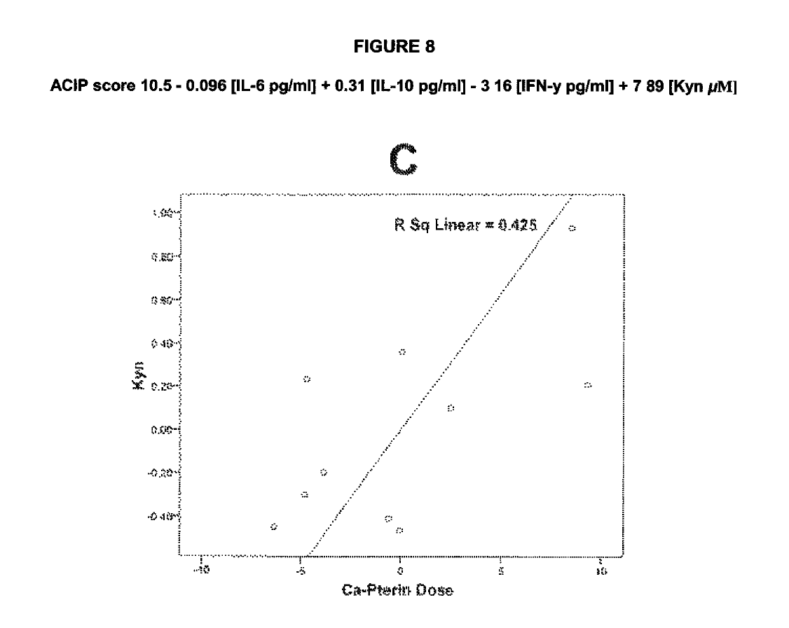

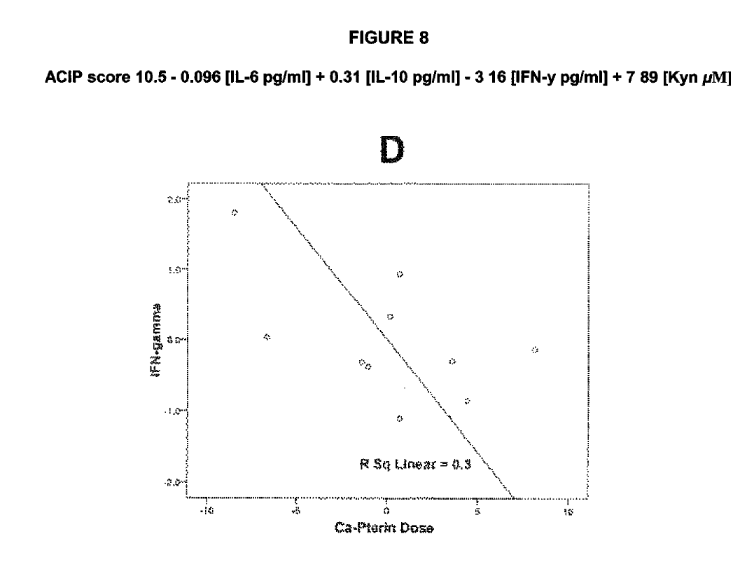

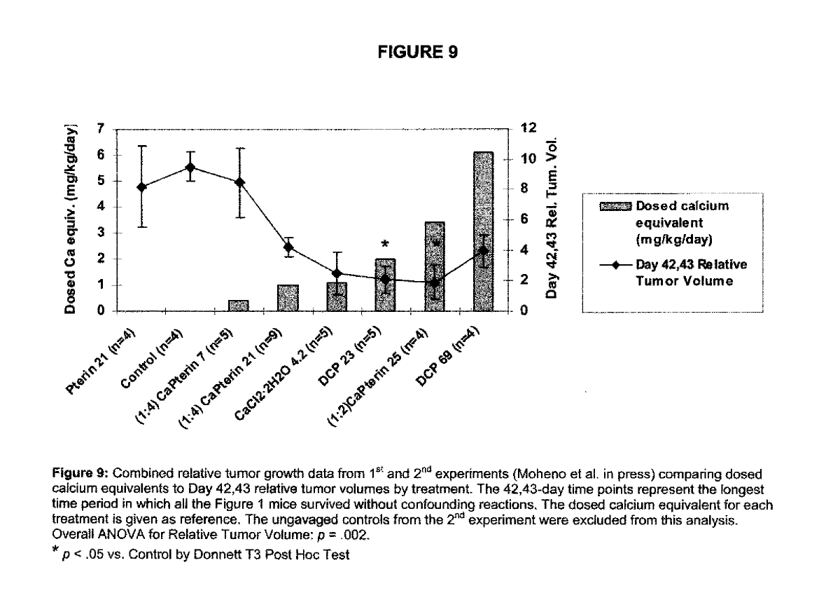

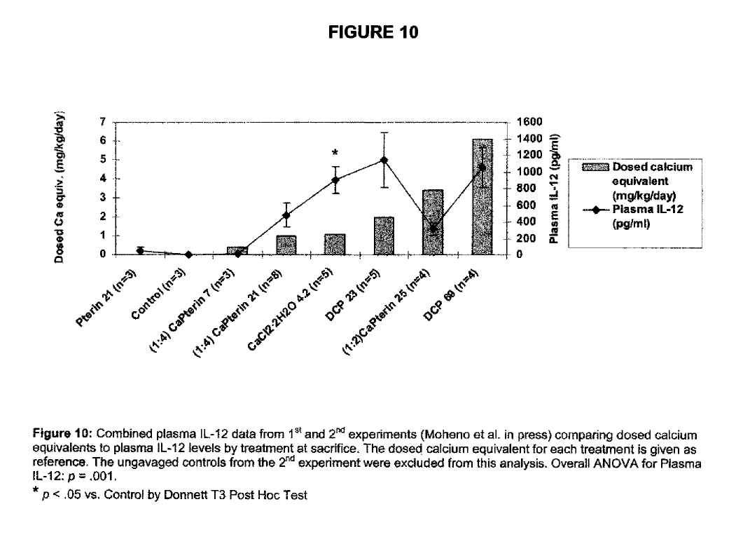

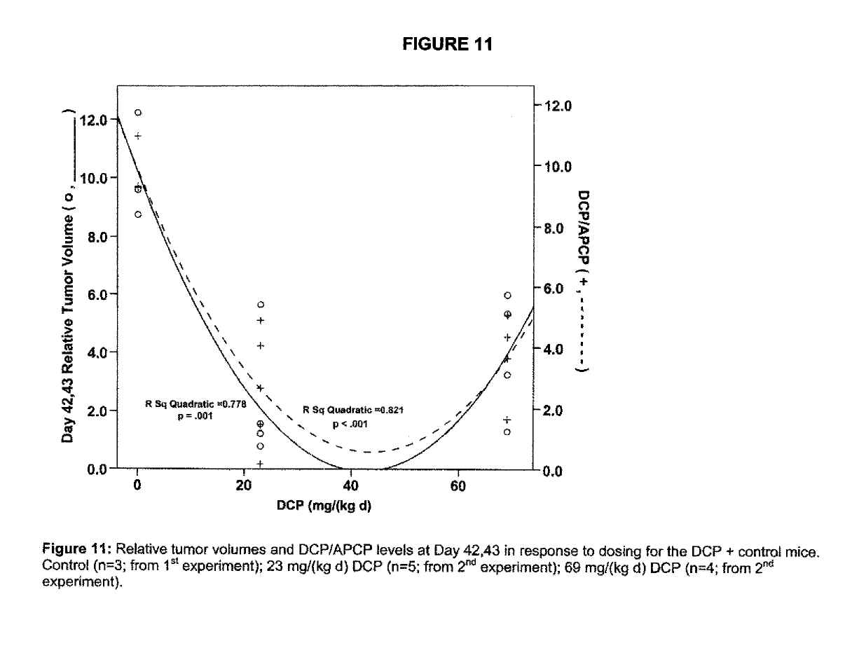

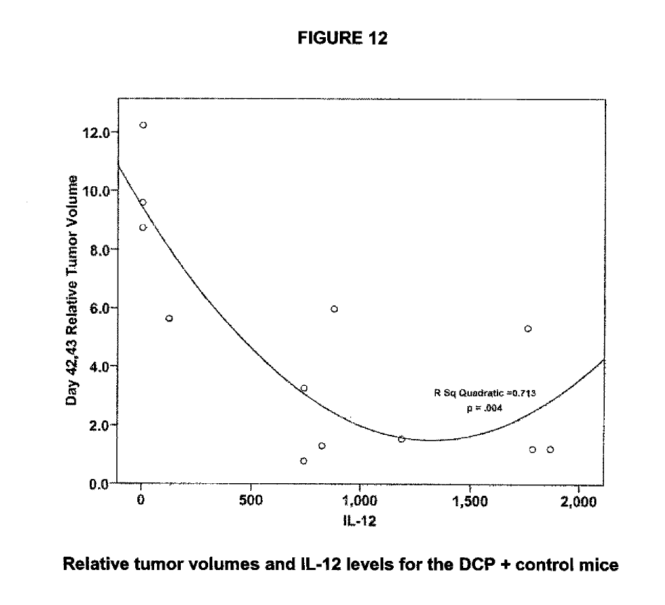

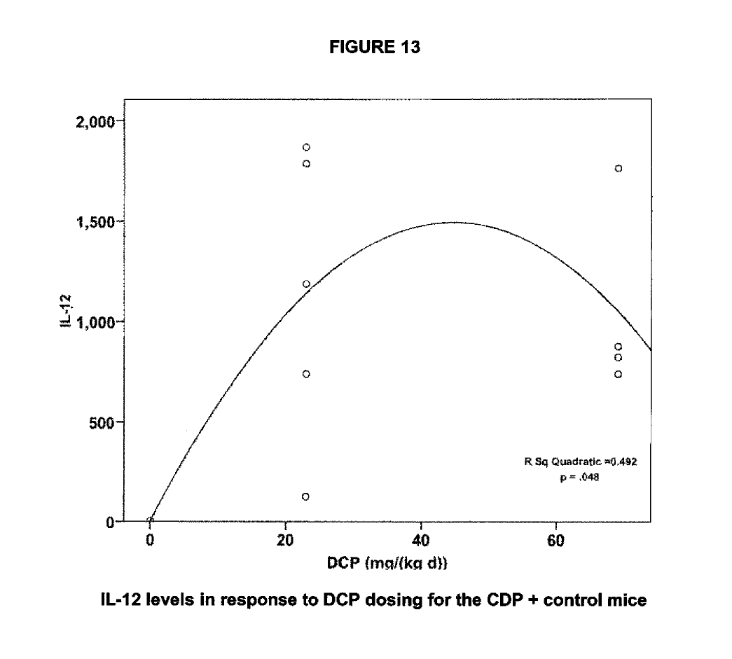

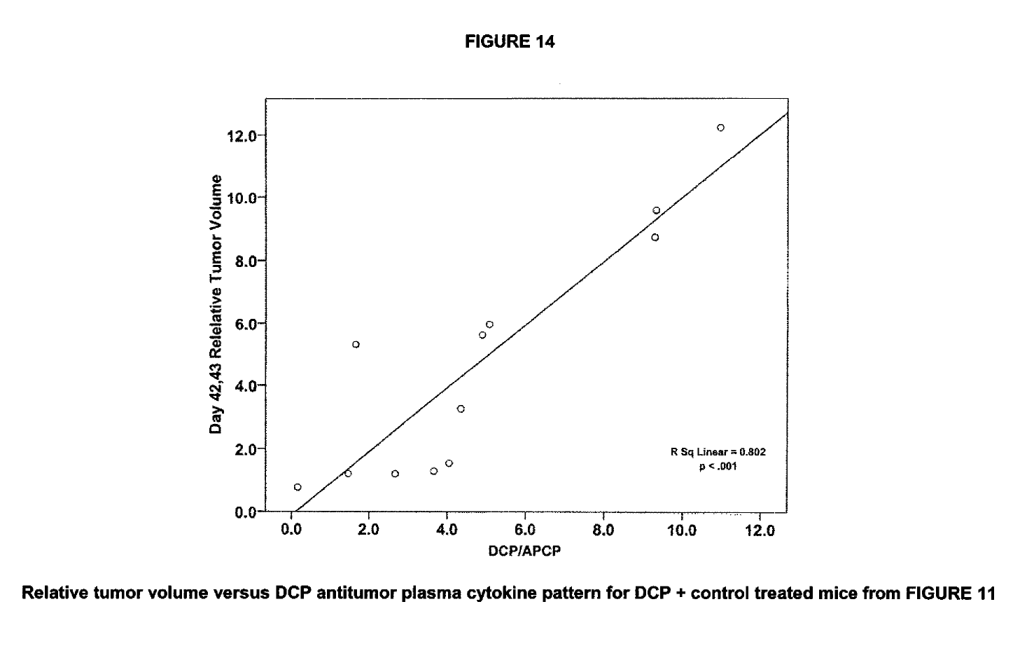

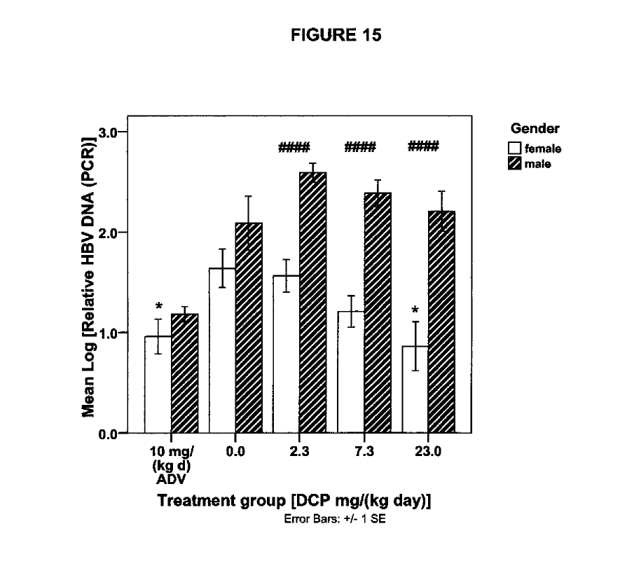

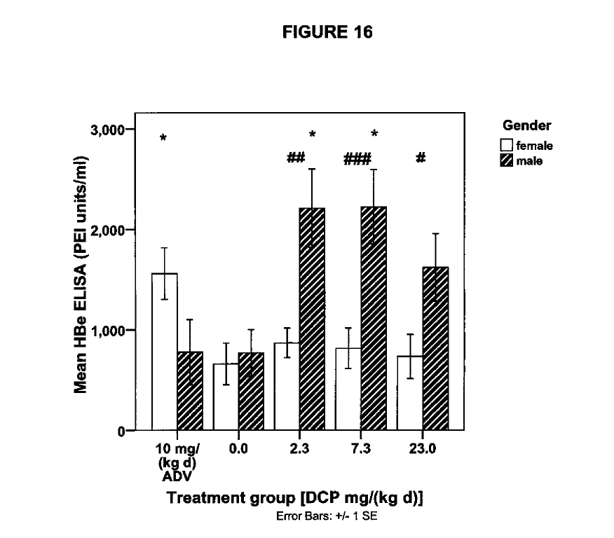

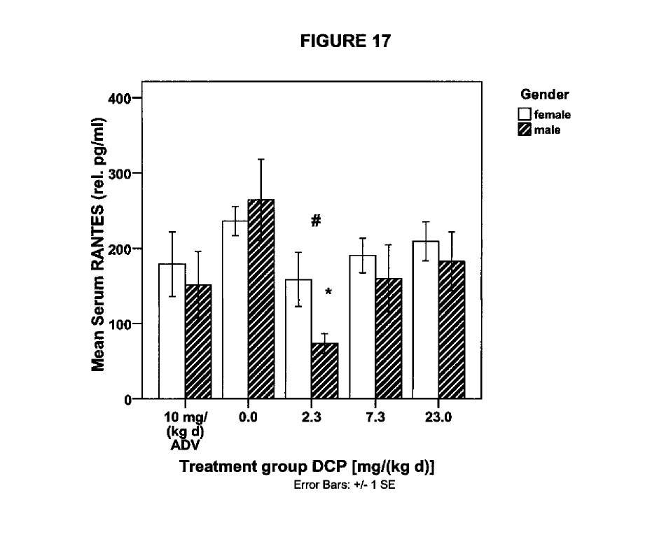

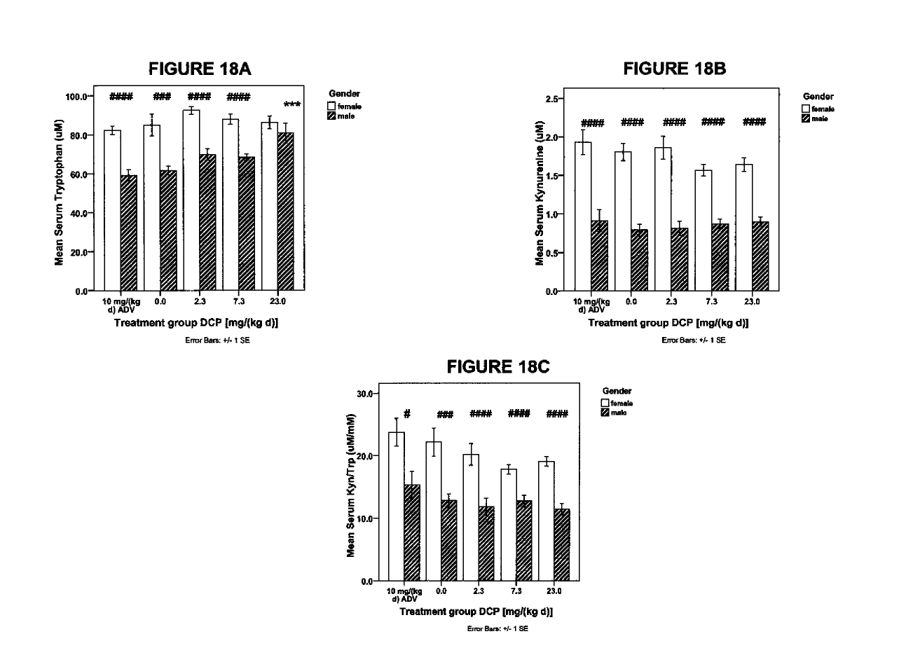

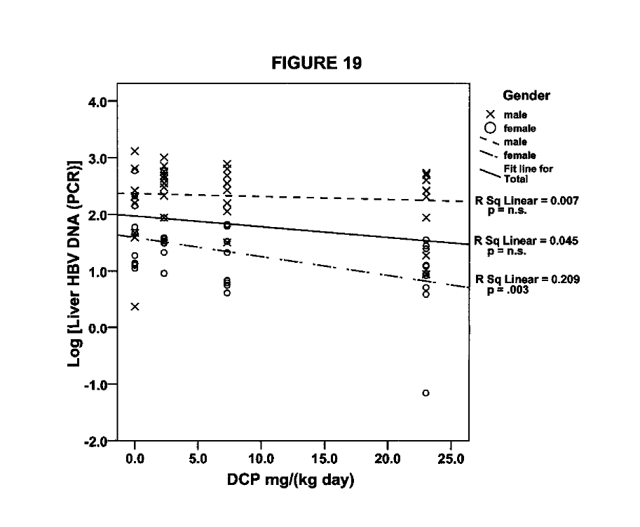

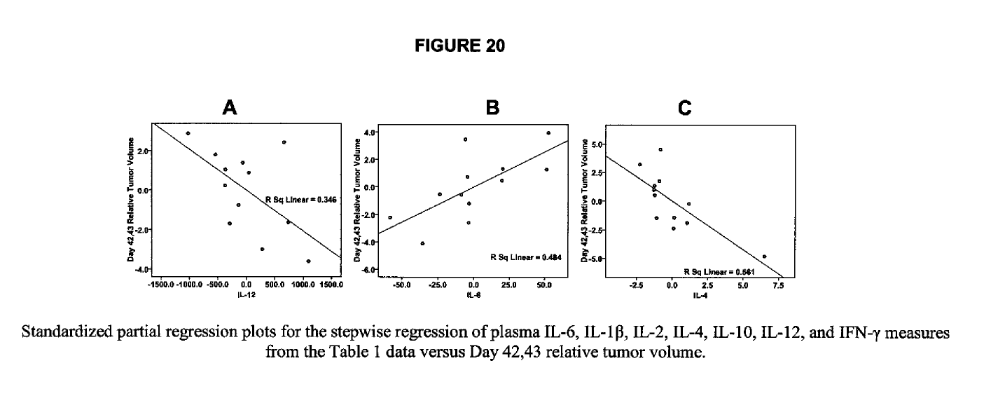

The question as to the role of calcium in mediating the efficacy of the various calcium-pterin complexes can be approached by plotting the relative tumor volumes for each treatment group, and the form of the dosed calcium-pterin complex, with the dosed calcium equivalent in each complex using the data in the 1^{st} and 2^{nd} experiments (FIG. 7). Over a 6-fold dosing range, from 1 to 6 mg/kg/day calcium equivalents, comparable antitumor efficacy is apparent among the various calcium forms. The rodent diet given the mice provided an additional 1,200 mg/kg/day of calcium, predominantly in the form of calcium carbonate, and to a much lesser extent calcium pantothenate (230 μg/kg/day calcium) and calcium iodate (12 μg/kg/day calcium). Therefore, calcium complexed with pterin, as well as calcium chloride dihydrate, possess antitumor activity not conferred by calcium complexed with carbonate.

Possible explanations for the unexpected tumor growth inhibition observed in the nude mice given calcium chloride dihydrate are that unchelated Ca⁺² 1) might in some way enhance the antitumor activity of the immune system of the mice, or 2) might have a direct effect upon the MDA-MB-231 breast cancer cells. A third possible explanation is that Ca⁺² ingestion leads to the formation of endogenous calcium pterin in the nude mice. Mice are known to have high liver tetrahydrofolic acid (THF) levels, 6.7 times higher than humans (Johlin et al., 1987). Furthermore, it is known that THF produces pterin upon acid oxidation (Blair et al., 1974). Orally ingested Ca⁺² ions in the unchelated form (i.e., CaCl₂) going from the intestinal tract directly to the liver can lower the pH of the liver and, within the oxidizing tissue environment of the liver, generate pterin from THF which can, in turn, form calcium pterin. This possible explanation can be tested through bioavailability studies measuring mouse plasma pterin changes in response to Ca⁺² ingestion. Further bioavailability studies can also closely assess the stability of calcium pterin complexes and DCP, and by determining their associated plasma clearance rates and metabolic products, identify bioactive forms.

A significant synergy exists between calcium and pterin in their ability to inhibit IDO in both unstimulated and PHA-stimulated human PBMCs (Table 3). Also, DCP is significantly more active than either CaCl₂ or pterin when tested individually. The enhanced IDO inhibition resulting from calcium and pterin synergy in CaPterin is comparable to that found with DCP. Taken together, these in vitro studies support the conclusion that calcium-complexed pterin forms (Ca-pterin and DCP) are more bioactive than their components, calcium and pterin, alone. Comparison of the pterin-equiv as Ca-Pterin IC₅₀ values with the pterin levels of normal human blood show that normal blood pterin concentrations are >25,000 times lower, well below estimated therapeutic levels even if fully complexed with calcium. DCP at the highest dose tested in mice in this study, 69 mg/kg, yields the following theoretical body fluid level: [DCP]_{fluid}=69 mg/kg÷0.7 L/kg÷454.4 mg/mmol=220 μM. This [DCP]_{fluid}, estimating a therapeutic mouse body fluid level, is comparable to the in vitro IDO IC₅₀ values of 470 μM and 320 μM determined for DCP in human PBMCs (Table 3).

The identified ACIP (Antitumor Cytokine/IDO Pattern) in vivo effects of CaPterin dosing, decreased IL-6 and IFN-γ, and increased IL-10 and kynurenine, reveal a pattern of immunological and metabolic responses correlated with CaPterin's antitumor efficacy (FIG. 4 and Table 2), as follows:
1) An increase in plasma IL-10 correlates with CaPterin dosage. IL-10 has been shown to be a critical, pleiotropic cytokine with contradictory properties (Vicari and Trinchieri, 2004). IL-10 has been mostly observed to possess anti-inflammatory (Th2) properties, antagonizing several functions of antigen-presenting cells (APCs) including dendritic cells (DCs). Investigators have also found ample evidence that IL-10 has a stimulating role in B cell proliferation, as well as natural killer (NK) cell and CD8+ cytotoxic T cell activation. Mocellin et al (2005) in their review of the available IL-10 evidence conclude that the data appear to support the hypothesis that IL-10 might contribute to the immune-mediated rejection of cancer, at least under some circumstances.
2) A decrease in plasma IL-6 correlates with increasing CaPterin dosage. The inflammatory cytokine IL-6 is an identified regulator that directs a shift from innate to acquired immunity (Jones, 2005). This immunological switching involves differential control of leukocyte recruitment, activation, and apoptosis. Further study is needed to explain how a down-regulation of IL-6 might regulate this switching in the context of an antitumor response.
3) A decrease in plasma IFN-γ correlates with increasing CaPterin dosage. The pro-inflammatory cytokine IFN-γ induces the enzyme IDO in a variety of cells (Wirleitner et al., 2003) which in turn can inhibit the response of T-cells to mitogen stimulation (Schrocksnadel et al., 2006) thus implicating IDO as a tumor escape mechanism (Uyttenhove et al., 2003). The finding that IFN-γ decreases with CaPterin dosages corroborates the findings of (Winkler et al., 2006) that CaPterin inhibits IDO activation in stimulated human PBMCs most likely by decreasing IFN-γ. Strategies to inhibit the IDO pathway may assist in breaking tolerance to tumors, and might enhance the efficacy of immunotherapeutic strategies by removing IDO counter-regulatory inhibition of T-cell activation (Munn, 2006).
4) The increase in plasma kynurenine correlating with CaPterin dosage can be largely explained by previous findings showing that kynurenine plasma levels correlate with plasma tryptophan levels, both of which decrease with increased tumor load (Schrocicsnadel et al., 2006). In those mice dosed with CaPterin, as tumor growth is inhibited, tryptophan and kynurenine plasma levels rise in concert. Since it has been previously shown that in vitro (1:4 mol:mol) calcium pterin inhibits IDO in PBMCs as measured by their production of kynurenine (Winkler at al., 2006), it is likely that in the lymphocytic microenvironment IDO is inhibited by calcium pterin, while at the systemic level kynurenine concentrations rise with tumor shrinkage due to the reduced tumor cell demands for circulating tryptophan. The resulting greater concentrations of available plasma tryptophan increase the substrate available to systemic IDO, thereby increasing plasma kynurenine levels as well.

The cytokine plasma changes caused by CaPterin can be generally understood as inducing sustained T-cell activity via IDO inhibition and the modulation of the inflammatory (Th1) immunological system. In addition, CaPterin can also sustain anti-inflammatory (Th2) activity via increased plasma IL-10. Th1 activity, modulated by the inhibition of IDO via decreased IFN-γ, leads to increased T-cell functioning, while Th2 anti-inflammatory activity is sustained by IL-10, which the regression analysis shows is increased by calcium pterin. IL-6, decreased by calcium pterin, reportedly plays a complex switching role between innate and acquired immunities. Significantly, in the context of chronic disease, the blocking of IL-6 signaling is proving to be therapeutically beneficial (Jones, 2005).

In conclusion, our results show that several forms of oral calcium pterin can inhibit MDA-MB-231 xenograph tumors in nude mice. Furthermore, a stepwise regression analysis of plasma cytokine and indoleamine 2,3-dioxygenase (IDO) metabolite levels show four effects correlated with (1:4 mol:mol) calcium pterin dosage: 1) decreased IL-6, 2) increased IL-10, 3) decreased IFN-γ, and 4) increased kynurenine. These findings imply a sustaining effect by calcium pterin of certain inflammatory and anti-inflammatory immunological responses.

An analysis of plasma cytokine changes resulting from the oral dosing of the antitumor agent (1:4 mol:mol) calcium pterin (CaPterin) found that it increased plasma IL-10, decreased plasma IL-6, and decreased plasma IFN-γ in nude mice with MDA-MB-231 xenograph tumors (Moheno et al. in press; 1^{st} experiment). The 2^{nd} experiment of this study found that a novel form of calcium pterin, dipterinyl calcium pentahydrate (DCP), along with CaCl₂.2H₂O, and (1:2 mol:mol) calcium pterin, all exhibited antitumor properties in this mouse-tumor system as well. Primarily due to the structural characteristics of DCP, substantial interest has been generated to elucidate its immunological effects, as was done with CaPterin. Therefore, in order to identify those plasma cytokine changes associated with tumor growth inhibition and arising from dosing with DCP, the following plasma cytokines in the nude mice from the 2^{nd} experiment (Moheno et al. in press) were measured: IL-1β, IL-2, IL-4, IL-5, IL-6, IL-10, IL-12, IFN-γ, TNF-α and TGF-β1.

### 5. MATERIALS AND METHODS

### 5.1. Test Substances

The following test substances were prepared as previously described (Moheno et al. in press):

(1:4 mol:mol) Calcium pterin suspension [CaPterin] (1 mg/ml);

Pterin suspension (1 mg/ml);

(1:2 mol:mol) Calcium pterin suspension (1.2 mg/ml);

Dipterinyl calcium pentahydrate (DCP) synthesis and suspensions; and

CaCl₂.2H₂O solution (0.2 mg/ml).

### 5.2. In Vivo Testing

5.2.1. Protocol

5.2.1.1 1^{st} Experiment

The protocol for the 1^{st} experiment has been described previously (Moheno et al. in press). Briefly, the aims of the 1^{st} experiment were to determine a dose-response curve for the (1:4 mol:mol) calcium pterin suspension and to compare the antitumor activity of this suspension to pterin alone (pterin control). Antitumor efficacy was evaluated in nude mice with MDA-MB-231 human tumor xenographs (Table 1).

5.2.1.2 2^{nd} Experiment

The protocol for the 2^{nd} experiment has also been described previously (Moheno et al. in press). Briefly, the aims of the 2^{nd} experiment were three-fold: 1) to test the antitumor effect of the increased [Ce⁺²] in the (1:2 mol:mol) calcium pterin suspension as compared to the (1:4 mol:mol) calcium pterin suspension; 2) to evaluate the antitumor efficacy of DCP at two concentrations, 23 mg/(kg day) and 69 mg/(kg day); and 3) to compare the antitumor activity of the calcium pterin to calcium chloride alone (CaCl₂ control) in athymic nude mice with MDA-MB-231 xenographs (Table 2).

In both experiments mice were treated by oral gavage once daily with the indicated test suspensions or solutions, with the following exception. The control mice in the 1^{st} experiment were treated with sterile water while the control mice in the 2^{nd} experiment were untreated (ungavaged controls) to evaluate the effect of mouse handling and gavaging upon tumor growth. Animals were restrained but not anesthetized for oral dosing. Tumors were measured twice weekly with calipers. Blood was collected from all animals via cardiac puncture at termination (after 70 to 98 days of treatment) and processed to EDTA plasma for analysis.

### 5.3. Measurements

5.3.1. Tumor Growth Rates

Each animal was individually tracked for tumor growth by external caliper measurements of protruding tumor. Primary tumor sizes were measured using calipers and an approximate tumor volume calculated using the formula 1/2 (a×b²), where b was the smaller of two perpendicular diameters.

5.3.2 Plasma Cytokines Levels

Cytokine levels in EDTA plasma from the mice in the 1^{st} experiment were determined by ELISA assay at Alta Analytical Laboratories (San Diego, Calif.) using a LINCOplex Kit (Linco Research).

Cytokine levels in EDTA plasma from the mice in the 2^{nd} experiment were measured at the UCSD Core Laboratory (San Diego, Calif.) using a multiplex assay kit for IL-1β, IL-2, IL-4, IL-5, IL-10, IL-12, IFN-γ, TNF-α and single ELISA kits for IL-6 and TGF-β1 by R&D Systems. EDTA plasma samples from two of the three ungavaged controls were lost due to handling error. For both the 1^{st} and 2^{nd} experiments, those measured cytokine values falling below the limits of detection were set to the higher assay limit for the purposes of the subsequent statistical analyses, and recorded as n.d. (not detectable) in Tables 1 through 3.

### 5.4. Statistics

Standard ANOVA models for group effects, curve fitting, and stepwise regression analyses were carried out using SPSS Graduate Pack 15.0 for Windows (2006), with p<0.05 used to determine significance.

### 6. RESULTS

Tables 4 and 5 give the mean plasma cytokine levels and standard errors (±SEM) at sacrifice for the nude mice from the 1^{st} and 2^{nd} experiments. TNF-α measures were excluded from further analyses since this variable failed the ANOVA test given in Table 3. The TNF-α measures were deemed to be unreliable because the 21 mg/(kg day) CaPterin-dosed mice from the 1^{st} and 2^{nd} experiments yielded significantly different plasma TNF-α values (p≦0.019). IL-5 and TGF-β1 were also excluded from subsequent analyses since they were not measured in the mice from the 1^{st} experiment (Table 4). All of the other plasma cytokine measures analyzed in Table 3 were found to be sufficiently uniform for statistical analysis (p>0.05).

TABLES 4 and 5
Mean plasma cytokine levels at sacrifice with standard errors (± SEM) for the
nude mice from the 1^{st} and 2^{nd} experiments are given. For comparability across the 1^{st} and 2^{nd}
experiments, the limit of detection for each plasma cytokine was set at the higher of the two limits
determined in the two experiments.

| | | CaPterin | Pterin | CaPterin | |
|---|---|---|---|---|---|
| Data from | Limits of | (7 mg/kg/d) | (21 mg/kg/d) | (21 mg/kg/d) | Control |
| 1^{st} experiment | detection | (n = 3) | (n = 3) | (n = 4) | (n = 3) |
| IL-6 (pg/ml) | 3.9 | 114.7 ± 43.2 | 87.4 ± 11.0 | 121.7 ± 78.1 | 104.5 ± 10.9 |
| TGF-β1 (pg/ml) | 31.2 | | | | |
| IL-1β (pg/ml) | 4.1 | 5.8 ± 1.7 | 64.6 ± 60.4 | n.d. | n.d. |
| IL-2 (pg/ml) | 12.6 | n.d. | n.d. | n.d. | n.d. |
| IL-4 (pg/ml) | 3.2 | n.d. | n.d. | n.d. | n.d. |
| IL-5 (pg/ml) | 3.0 | | | | |
| IL-10 (pg/ml) | 24.6 | 49.0 ± 17.0 | n.d. | 61.5 ± 27.3 | n.d. |
| IL-12 (pg/ml) | 3.2 | 7.7 ± 4.4 | 48.5 ± 45.3 | 243.8 ± 237.8 | n.d. |
| IFN-γ (pg/ml) | 3.2 | 4.7 ± .8 | n.d. | n.d. | n.d. |
| TNF-α (pg/ml) | 3.2 | 9.8 ± 1.5 | 9.0 ± 3.7 | 8.5 ± 1.6 | 4.3 ± 0.5 |

| | | | [1:2] | | | | Ungavaged/ |
|---|---|---|---|---|---|---|---|
| Data from | Limits | CaPterin | CaPterin | DCP | DCP | | Untreated |
| 2^{nd} | of | (21 mg/kg/d) | (25 mg/kg/d) | (23 mg/kg/d) | (69 mg/kg/d) | CaCl₂•2H₂O | Control |
| experiment | detection | (n = 4)^{b} | (n = 4)^{b} | (n = 5)^{b} | (n = 4)^{b} | (n = 5)^{b} | (n = 1) |
| IL-6 (pg/ml) | 3.9 | 30.3 ± 10.2 | 31.6 ± 9.2 | 49.6 ± 18.5 | 37.0 ± 11.3 | 44.4 ± 15.4 | 18.6 |
| TGF-β1 (pg/ml) | 31.2 | n.d. | n.d. | n.d. | 49.5 ± 18.3 | 74.3 ± 35.0 | 45.3 |
| IL-1β (pg/ml) | 4.1 | 5.0 ± .7 | 4.9 ± .5 | 6.2 ± .8 | 5.3 ± .7 | 6.4 ± .9 | 21.3 |
| IL-2 (pg/ml) | 12.6 | n.d. | n.d. | 12.7 ± .11 | n.d. | 12.7 ± .13 | n.d. |
| IL-4 (pg/ml) | 3.2 | n.d. | n.d. | 5.7 ± 1.7 | 4.0 ± .6 | 3.6 ± .3 | n.d. |
| IL-5 (pg/ml) | 3.0 | 8.2 ± 1.4 | 4.4 ± .9 | 20.3 ± 10.2 | 24.9 ± 17.0 | 10.3 ± 2.4 | 37.5 |
| IL-10 (pg/ml) | 24.6 | n.d. | 30.2 ± 5.6 | 37.0 ± 12.4 | n.d. | 26.5 ± 1.9 | n.d. |
| IL-12 (pg/ml) | 3.2 | 721.6 ± 19.1 | 315.2 ± 71.0 | 1141.7 ± 327.1 | 1050.52 ± 238.7 | 904.9 ± 156.9 | 717.0 |
| IFN-γ (pg/ml) | 3.2 | 6.2 ± 2.4 | 16.1 ± 12.9 | 15.2 ± 4.9 | 29.9 ± 18.0 | 9.7 ± 3.3 | 7.0 |
| TNF-α (pg/ml) | 3.2 | n.d. | n.d. | n.d. | n.d. | n.d. | n.d. |

n.d. = not detectable

FIG. 9 shows that nude mice dosed with 23 mg/(kg day) DCP and 25 mg/(kg day) (1:2 mol:mol) calcium pterin showed significant tumor growth inhibition relative to controls. FIG. 10 shows significantly elevated plasma IL-12 for 4.2 mg/(kg day) CaCl₂.2H₂O. ANOVA analyses of the other plasma cytokine levels found that none differed significantly from controls for any of the treatment groups.

Table 7 gives the plasma cytokine and relative tumor volume Spearman rank order correlations for the 12 DCP+ control mice (Control [n=3; from 1^{st} experiment]; 23 mg/(kg d) DCP [n=5; from 2^{nd} experiment]; and 69 mg/(kg d) DCP [n=4; from 2^{nd} experiment]). The correlations identify IL-12, IL-1b, and IL-4 as significant inverse correlates to relative tumor volume, with IL-12 and IL-1b significantly intercorrelated.

To further determine significant causal and correlational linkages among DCP dosage, plasma cytokine changes, and relative tumor volume, the following curve fitting analyses were carried out with the 12 DCP+ control mice. First, plotting DCP versus Day 42,43 Relative Tumor Volume yielded a significant quadratic relationship (FIG. 11). Second, curve fitting each cytokine versus Day 42,43 Relative Tumor Volume found no significant linear relationship and only one significant quadratic relationship, i.e., with IL-12 (p=0.004; FIG. 12). Third, plotting DCP versus IL-12 yielded a weakly significant quadratic relationship (p=0.048; FIG. 13).

Substantially more significant analogous plots were generated by first carrying out the Table 8 stepwise regression analysis which derived the DCP antitumor plasma cytokine pattern (DCP/APCP), shown to be a more significant cytokine composite measure than IL-12 taken alone. The Table 8 stepwise regression analysis of the cytokine data from the 12 DCP+ control mice allowed for the calculation of DCP/APCP scores for each mouse. These DCP/APCP scores are plotted versus DCP dosage in FIG. 11, which illustrates the significant quadratic dose-response relationships of DCP versus the derived plasma DCP/APCP composite measure. From this figure the estimated optimum dosage of DCP for maximum tumor growth inhibition is 42 mg/(kg day), while the estimated optimum dosage for maximum DCP/APCP induction is 44 mg/(kg day). FIG. 14 shows the strongly significant relationship between relative tumor volume and the DCP/APCP composite measure for the 12 DCP+ control mice from FIG. 11.

TABLE 7
Rank Order Correlations for the DCP + control mice.

| | | | Day 42, | | | | | | | |
|---|---|---|---|---|---|---|---|---|---|---|
| | | | 43 | | | | | | | |
| | | | Relative | | | | | | | |
| | | DCP | Tumor | | | | | | | |
| Spearman's rho | | (mg/kg/d) | Volume | IL-6 | IL-1b | IL-2 | IL-4 | IL-10 | IL-12 | IFN-γ |
| DCP | Correlation | 1.000 | | | | | | | | |
| (mg/kg/d) | Coefficient | | | | | | | | | |
| | Sig. (2- | — | | | | | | | | |
| | tailed) | | | | | | | | | |
| | N | 12 | | | | | | | | |
| Day42, 43 | Correlation | −.402 | 1.000 | | | | | | | |
| Relative | Coefficient | | | | | | | | | |
| Tumor | Sig. (2- | .195 | — | | | | | | | |
| Volume | tailed) | | | | | | | | | |
| | N | 12 | 12 | | | | | | | |
| IL-6 | Correlation | −.544 | .294 | 1.000 | | | | | | |
| | Coefficient | | | | | | | | | |
| | Sig. (2- | .068 | .354 | — | | | | | | |
| | tailed) | | | | | | | | | |
| | N | 12 | 12 | 12 | | | | | | |
| IL-1b | Correlation | .394 | −.631(*) | −.102 | 1.000 | | | | | |
| | Coefficient | | | | | | | | | |
| | Sig. (2- | .205 | .028 | .754 | — | | | | | |
| | tailed) | | | | | | | | | |
| | N | 12 | 12 | 12 | 12 | | | | | |
| IL-2 | Correlation | −.047 | −.480 | .131 | .226 | 1.000 | | | | |
| | Coefficient | | | | | | | | | |
| | Sig. (2- | .886 | .114 | .685 | .479 | — | | | | |
| | tailed) | | | | | | | | | |
| | N | 12 | 12 | 12 | 12 | 12 | | | | |
| IL-4 | Correlation | .293 | −.591(*) | .266 | .496 | .572 | 1.000 | | | |
| | Coefficient | | | | | | | | | |
| | Sig. (2- | .356 | .043 | .403 | .101 | .052 | — | | | |
| | tailed) | | | | | | | | | |
| | N | 12 | 12 | 12 | 12 | 12 | 12 | | | |
| IL-10 | Correlation | −.047 | .131 | −.480 | −.317 | −.091 | −.208 | 1.000 | | |
| | Coefficient | | | | | | | | | |
| | Sig. (2- | .886 | .685 | .114 | .315 | .779 | .517 | — | | |
| | tailed) | | | | | | | | | |
| | N | 12 | 12 | 12 | 12 | 12 | 12 | 12 | | |
| IL-12 | Correlation | .540 | −.704(*) | −.416 | .818(**) | −.044 | .277 | −.220 | 1.000 | |
| | Coefficient | | | | | | | | | |
| | Sig. (2- | .070 | .011 | .179 | .001 | .892 | .384 | .492 | — | |
| | tailed) | | | | | | | | | |
| | N | 12 | 12 | 12 | 12 | 12 | 12 | 12 | 12 | |
| IFN-γ | Correlation | .236 | −.197 | .338 | .518 | .308 | .436 | −.484 | .326 | 1.000 |
| | Coefficient | | | | | | | | | |
| | Sig. (2- | .460 | .539 | .283 | .084 | .330 | .157 | .111 | .301 | — |
| | tailed) | | | | | | | | | |
| | N | 12 | 12 | 12 | 12 | 12 | 12 | 12 | 12 | 12 |

(*)Correlation is significant at the 0.05 level (2-tailed).
(**)Correlation is significant at the 0.01 level (2-tailed).

TABLE 8
The DCP antitumor plasma cytokine pattern (DCP/APCP) was
derived from the stepwise regression of plasma IL-6, IL-
1β, IL-2, IL-4, IL-10, IL-12, and IFN-γ measures
from the Table 1 data versus Day 42, 43 relative tumor volume.

| | | Standard |
|---|---|---|
| | Regression Model | Regression |
| | Coefficients* | Coefficients* |
| CaPterin Dose | 7.235 (Intercept) | |
| Versus: | | |
| Plasma IL-12 | −0.002 | −0.382 |
| Plasma IL-6 | 0.051 | 0.531 |
| Plasma IL-4 | −0.846 | −0.572 |

Probability of F-to-Enter = 0.150 and Probability of F-to-Remove = 0.200 were set.
The model is significant to *p = .003. Standardized partial regression plots are given in FIG. 20.
DCP/APCP score = 7.235 − 0.002 [IL12 pg/ml] + 0.051 [IL6 pg/ml] − 0.846 [IL4 pg/ml]

### 7. DISCUSSION

FIG. 6 shows that the derived DCP/APCP composite measure strongly correlates with relative tumor growth in the context of the oral DCP dosing of nude mice with MDA-MB-231 xenograph tumors. The ability of DCP to quadratically correlate with both relative tumor volume and DCP/APCP, which defines optimal relative antitumor plasma levels for IL-12 (high), IL-6 (low), and IL-4 (high) (FIG. 11; Table 8), corroborates the finding that (1:4 mol:mol) calcium pterin (CaPterin) at lower dosages decreases plasma IL-6 in this mouse-tumor system (Moheno at al in press). DCP and CaCl₂.2H₂O exert their antitumor efficacy in a manner correlated to IL-12 induction in contrast to (1:4 mol:mol) calcium pterin and (1:2 mol:mol) calcium pterin which induced lower, non-significant increases in plasma IL-12 at the dosages tested here (FIG. 10).

The Table 7 Spearman rank order correlations among DCP dosings, cytokine measures, and relative tumor volumes also corroborate the findings that IL-12 and IL-4 correlate in a rank order manner with relative tumor volume. Since DCP plots as a quadratic relative tumor volume and DCP/ACPC effector (FIG. 11), significant linear rank order correlations are absent.

A recent review of IL-12-based immunotherapy for cancer (Weiss at al 2007) concludes that IL-12 holds considerable promise because it 1) is a regulator of both innate and adaptive immune responses, 2) can by itself induce potent anticancer effects, and 3) synergizes with several other cytokines for increased immunoregulatory and antitumor activities. The review further finds that as an antitumor cytokine, IL-12 has the ability to synergize with other cytokines to enhance immune effector cell populations and to regulate host-tumor cell interactions within the tumor microenvironment.

In conclusion, DCP induces a significant quadratic antitumor response correlated to increased plasma IL-12, decreased IL-6, and increased IL-4 which are optimized in the 40-46 mg/(kg day) dose range for nude mice with MDA-MB-231 xenograph tumors.

Activity of DCP against Hepatitis B Virus Infection

DCP also has utility as a therapy for the treatment of hepatitis B infection. DCP induces a significant dose-response reduction of Log liver HBV DNA (PCR) in female HBV mice. DCP also increased HBe antigen (ELISA) among male mice. However, DCP did not affect the serum concentrations of the IDO metabolites, tryptophan (Trp) and kynurenine (Kyn), and the Kyn/Trp ratio, except for tryptophan (Trp) at 23.0 mg/(kg day) among male HBV mice. Nevertheless, these three IDO-related measures were broadly elevated in female mice compared to male mice. The serum concentration of the chemokine RANTES was decreased in male HBV mice by 2.3 mg/(kg day) DCP. Serum cytokines, IL-4, IL-9, and were elevated by 7.3 mg/(kg day) DCP among females.

Immunomodulation via IDO or TDO (tryptophan 2,3-dioxygenase) pathways are proposed to be involved in the modulation of HBV expression in the transgenic mice and in the anti-HBV mechanism of DCP, based upon DCP's gender-specific inhibition of viral replication, and the correlation of elevated IDO metabolites with reduced viral parameters in female HBV mice independent of DCP-treatment.

Hepatitis B virus (HBV) causes both transient and persistent infections of the liver in humans. The number of chronic HBV carriers is estimated to be 400 million worldwide; nearly 25% of which are estimated will terminate in liver failure or liver cancer (Seeger C & Mason W S. Hepatitis B virus biology. Microbiol Mol Biol Rev 2000; 64:51-68). Dipterinyl calcium pentahydrate (DCP) has demonstrated significant antitumor activity associated with plasma IL-12 concentration increases in MDA-MB-231 (human breast cancer) xenographed nude mice (Moheno P, Pfleiderer W, Dipasquale A G, Rheingold A L & Fuchs D. Cytokine and IDO metabolite changes effected by calcium pterin during inhibition of MDA-MB-231 xenograph tumors in nude mice. Int J Pharm 2008; 355:238-248; Moheno P, Pfleiderer W & Fuchs D. Plasma Cytokine Concentration Changes Induced by the Antitumor Agents Dipterinyl Calcium Pentahydrate (DCP) and Related Calcium Pterins. Immunobiology in press).

### Methods

Animals. Homozygous adult female and male transgenic HBV mice were used (20.6±2.8 g). The mice were originally obtained from Dr. Frank Chisari (Scripps Research Institute, La Jolla, Calif.) (Guidotti L G, Matzke B, Schaller H & Chisari F V. High-level hepatitis B virus replication in transgenic mice. J Virol 1995; 69:6158-6169) and were subsequently raised in the Biosafety Level 3 (BL-3) area of the AAALAC-accredited USU Laboratory Animal Research Center (LARC). The animals were derived from founder 1.3.32 (Guidotti L G, Matzke B, Schaller H & Chisari F V. High-level hepatitis B virus replication in transgenic mice. J Virol 1995; 69:6158-6169). This study was conducted in accordance with the approval of the Institutional Animal Care and Use Committee of Utah State University.

Experimental design. DCP was administered per os, once daily for 14 days to randomly assigned HBV transgenic mice at 23, 7.3, and 2.3 mg/(kg d). ADV was used as a positive control at 10 mg/(kg d) using the same treatment schedule and vehicle (0.4% CMC). On day 14, mice were euthanized to collect serum and liver samples to perform liver HBV DNA, liver core and serum HBe assays, serum cytokine/chemokine profiles, and IDO metabolite assays.

### Methods: Chemistry

Compounds. DCP was suspended in 0.4% carboxymethylcellulose (CMC) at concentrations sufficient to deliver the desired dose in a volume of 0.1 mL per 20-g mouse. The solution was stored at 4° C. during the course of the experiment. The volume was adjusted for the weight of each mouse. The structure of DCP is given in reference (Moheno P, Pfleiderer W, Dipasquale A G, Rheingold A L & Fuchs D. Cytokine and IDO metabolite changes effected by calcium pterin during inhibition of MDA-MB-231 xenograph tumors in nude mice. Int J Pharm 2008; 355:238-248). A positive control, adefovir dipivoxil (ADV) (Gilead Pharmaceuticals) was prepared in the same manner as the DCP for the appropriate dosage.

### Methods: Virology

Liver HBV DNA assays. Southern blot hybridization and quantitative PCR (qPCR) were performed on liver tissues (Morrey J D, Motter N E, Tam B, Lay M & Fairman J. Efficacy of cationic lipid-DNA complexes (CLDC) on hepatitis B virus in transgenic mice. Antiviral Res 2008; 79:71-79). For Southern blot hybridization, the ratio of the viral DNA bands to the transgene band was used to determine the concentration of viral DNA per host DNA. This calculation was based upon the knowledge that there were 1.3 copies of the transgene present per host cell with this line of transgenic mice. The transgene was used as an internal indicator to calculate the pg of HBV DNA per μg of homozygous cellular host DNA. For qPCR, the assay was run with a series of 10-fold dilutions of pooled liver DNA from HBV transgenic mice to obtain a standard curve. Mean C(t) values were obtained for duplicates of each sample. The mean C(t) values of each sample were used to obtain the log relative DNA values using a formula of the fit line of the standard curve.

Liver or serum cytokine/chemokine array. Liver samples were prepared for the Q-Plex™ mouse cytokine/chemokine array (Quansys Biosciences, Logan, Utah) as described previously (Morrey J D, Molter N E, Taro B, Lay M & Fairman J. Efficacy of cationic lipid-DNA complexes (CLDC) on hepatitis B virus in transgenic mice. Antiviral Res 2008; 79:71-79).

Sera HBeAg. Whole blood samples were obtained during necropsy by cardiac puncture, and processed for an HBeAg-specific ELISA (International Immuno Diagnostics, Foster City Calif.) (Money J D, Motter N E, Taro B, Lay M & Fairman J. Efficacy of cationic lipid-DNA complexes (CLDC) on hepatitis B virus in transgenic mice. Antiviral Res 2008; 79:71-79). Using the known PEI (Paul Ehrlich Institute) units for the calibrator, PEI units were formulated for the serial dilutions of the positive serum. A graph was generated, and extrapolation was used to assign a PEI unit value for each sample.

Liver HBcAg assay. Liver biopsies were processed for detection of hepatitis B core antigen (HBcAg) (Motley J D, Motter N E, Taro B, Lay M & Fairman J. Efficacy of cationic lipid-DNA complexes (CLDC) on hepatitis B virus in transgenic mice. Antiviral Res 2008; 79:71-79). Three different parameters were obtained from each tissue section. The first two measurements are based on the observation that cells surrounding the central veins of the liver are more strongly stained than are other areas of the liver (personal observation). The first two parameters were obtained by counting cells surrounding central veins as follows: the total number of cells, the number of cells with stained nuclei, and the number of cells with stained cytoplasms. The identities of the samples were blinded to person counting. The stained nuclei counts or the stained cytoplasm counts were divided by the total cells. Three central vein areas were counted for each slide sample. For the third parameter, a field not in a central vein area was counted for the total number of stained nuclei. One-quarter of the field was counted. Three such fields were counted per liver section. The identity of the samples was blinded to the person reading the slides.

Tryptophan and kynurenine measurements. Tryptophan (Trp) and kynurenine (Kyn) measurements were carried out as previously described (Widner B, Werner E R, Schennach H, Wachter H & Fuchs D. Simultaneous measurement of serum tryptophan and kynurenine by HPLC. Clin Chem 1997; 43:2424-2426). Kyn/Trp ratios were calculated for each mouse as an estimate of IDO activity.

Statistical analysis. Analyses were carried out using SPSS Graduate Pack 15.0 for Windows (2006), with p<0.05 used to determine significance. Those measures found to be significant by the Kruskal-Wallis nonparametric test for treatment group effects were then tested by one-way ANOVA, followed by post-hoc 2-sided Dunnett tests (for equal variances) or Dunnett's T3 tests (for unequal variances) versus controls. The Mann-Whitney U nonparametric test was used to test gender effects, followed by one-way ANOVA.

### Results

### Statistical Analysis

The following viral, IDO, and cytokine/chemokine measures were collected in this study: liver HBV DNA (Southern), liver HBV DNA (PCR), HBe antigen (ELISA), Average #HBcAg Nuclei, Average #HBcAg Cytoplasms, #HBcAg Nuclei per Quarter Field; serum Tryptophan, Kynurenine, Kyn/Trp, IL-1a, IL-1b, IL-2, IL-3, IL-4, IL-6, IL-9, IL-10, IL-12, MCP-1, TNF-α, MIP-1, GM-CSF, RANTES; and liver IL-6. FIG. 15 gives the summary statistics for Log HBV DNA (PCR) by treatment group and gender showing significant treatment decreases for female HBV mice with 23.0 mg/(kg day) DCP and ADV, and significant gender differences with 2.3, 7.3, and 23.0 mg/(kg day) DCP. FIG. 16 gives the summary statistics for HBe antigen (ELISA) by treatment group and gender showing significant treatment increases for the males with 2.3 and 7.3 mg/(kg day) DCP, and a significant increase for the females with ADV. Significant gender differences in HBe antigen are found for DCP dosings at 2.3, 73, and 23.0 mg/(kg day) DCP. The viral measures not showing significant differences from the treatment controls [liver HBV DNA (Southern), Average #HBcAg Nuclei, Average #HBcAg Cytoplasms, #HBcAg Nuclei per Quarter Field] are not graphed.

The serum chemokine RANTES, showed a significant decrease in the male HBV mice at 2.3 mg/(kg day) DCP (FIG. 17), as well as a significant gender difference at the same DCP dosage.

The other significant gender effects for viral and cytokine serum measures are given in Table 9. IL-4, IL-9, and IL-12 were significantly elevated at 7.3 mg/(kg day) DCP in female mice as compared to male mice. This cytokine elevation was associated with reduced liver HBV DNA in female mice compared to male mice (FIG. 19).

Treatment group and gender effects are graphed for Trp, Kyn, and Kyn/Trp in FIGS. 18A, 18B, and 18C. DCP significantly increased serum tryptophan (Trp) only at the highest dosage tested, 23.0 mg/(kg day) in male HBV mice, but DCP did not affect the serum concentrations of the other IDO metabolite measures, Kyn, and Kyn/Trp for the mice generally. Importantly however, Trp, Kyn, and Kyn/Trp were broadly elevated in female HBV transgenic mice as compared to the male mice.

### Discussion

FIG. 15 shows that the highest dose of DCP tested, 23.0 mg/(kg day), and ADV both significantly lowered liver HBV DNA in female mice. Moreover, FIG. 15 shows that, generally, the anti-HBV efficacy of DCP is greater for the female HBV mice than for the males. FIG. 19 gives the three plots of Log [HBV DNA (PCR)] versus DCP dosage 1) for the female HBV mice (p=0.003; R²=0.209), 2) for the male HBV mice (p=n.s.; R²=0.007), and 3) for all the mice (p=n.s.; R²=0.045). The significant regression equation from this graph for the female HBV mice is:

Log [Liver HBV DNA (PCR)]=1.59−0.033 DCP

By linear extrapolation from this equation, a DCP dosage of 90 mg/(kg day) might be expected to lead to a 3 Log reduction in liver HBV DNA, as measured by PCR, in the female HBV mice.

Notably, the mean control female HBV mouse serum Kyn/Trp ratio (22.2±2.2 uM/mM) is closer in magnitude to normal human serum Kyn/Trp (26.5−45.0 uM/mM) (Weinlich G, Murr C, Richardsen L, Winkler C & Fuchs D. Decreased serum tryptophan concentration predicts poor prognosis in malignant melanoma patients. Dermatology 2007; 214:8-14; Frick B, Schroecksnadel K, Neurauter G, Leblhuber F & Fuchs D. Increasing production of homocysteine and neopterin and degradation of tryptophan with older age. Clin Biochem 2004; 37:684-687; Widner B, Leblhuber F, Walli J, Tilz G P, Demel U & Fuchs D. Tryptophan degradation and immune activation in Alzheimer's disease. J Neural Transm 2000; 107:343-353) than is the mean control serum Kyn/Trp for male HBV mice (12.8±1.1 uM/mM). Thus, based upon estimated serum IDO activity (Trp/Kyn) and the FIG. 19 dose-response plots, the female HBV mice may be a more suitable model than the males in which to demonstrate DCP's anti-HBV efficacy, should this efficacy is linked to IDO inhibition in mammals with high IDO serum levels.

The observation that female transgenic mice have significantly higher serum Trp, Kyn, and Trp/Kyn (an estimate of IDO activity) levels (FIG. 18) than the male mice may be biologically relevant to HBV expression in these transgenic mice. IDO immuno-inhibition appears to take two forms, 1) as a depletor of the nutrient tryptophan, and 2) through the direct action of its enzymatic products, kynurenine, 3-hydroxykynurenine, and 3-hydroxyanthranilic acid (Zamanakou M, Germenis A E & Karanikas V. Tumor immune escape mediated by indoleamine 2,3-dioxygenase. Immunol Lett 2007; 111:69-75; Temess P, Bauer T M, Rose L, Dufter C, Watzlik A, Simon H & Opelz G. Inhibition of allogeneic T cell proliferation by indoleamine 2,3-dioxygenase-expressing dendritic cells: mediation of suppression by tryptophan metabolites. J Exp Med 2002; 196:447-457; Weinlich G, Murr C, Richardsen L, Winkler C & Fuchs D. Decreased serum tryptophan concentration predicts poor prognosis in malignant melanoma patients. Dermatology 2007; 214:8-14; Brandacher G, Cakar F, Winlder C, Schneeberger S, Obrist P, Bosmuller C, Werner-Felmayer G, Werner E R, Bonatti H, Margreiter R & Fuchs D. Non-invasive monitoring of kidney allograft rejection through IDO metabolism evaluation. Kidney Int 2007; 71:60-67; Frumento G, Rotondo R, Tonetti M, Damonte G, Benatti U & Ferrara G B. Tryptophan-derived catabolites are responsible for inhibition of T and natural killer cell proliferation induced by indoleamine 2,3-dioxygenase. J Exp Med 2002; 196:459-468). In previous studies, female HBV mice were identified to have slightly lower levels of liver HBV DNA than male mice (Morrey J D, Korba B E & Sidwell R W. Transgenic mice as a chemotherapeutic model for HBV infection. In Therapies for Viral Hepatitis. Edited by Editor|. Year|; p.̂pp. Pages|. City|: Publisher|; Julander J G, Colonno R J, Sidwell R W & Morrey J D. Characterization of antiviral activity of entecavir in transgenic mice expressing hepatitis B virus. Antiviral Research 2003; 59:155-161) (FIG. 15). These two gender differences, the IDO activity and liver HBV DNA levels, might have related mechanisms. For example, HBV surface antigens can be regulated in HBV transgenic mice by sex steroids and glucocorticoids (Farza H, Salmon A M, Hadchouel M, Moreau J L, Babinet C, Tiollais P & Pourcel C. Hepatitis B surface antigen gene expression is regulated by sex steroids and glucocorticoids in transgenic mice. Proc. Natl. Acad. Sci USA 1987; 84:1187-1191). Similarly, both IDO, and its related hepatic enzyme TDO (hepatic tryptophan pyrrolase; tryptophan 2,3-dioxygenase) can be upregulated by steroids like estrogen (Danesch U, Gloss B, Schmid W, Schutz G, Schule R & Renkawitz R. Glucocorticoid induction of the rat tryptophan oxygenase gene is mediated by two widely separated glucocorticoid-responsive elements. Embo J 1987; 6:625-630; Grohmann U, Volpi C, Fallarino F, Bozza S, Bianchi R, Vacca C, Orabona C, Belladonna M L, Ayroldi E, Nocentini G, Boon L, Bistoni F, Fioretti M C, Romani L, Riccardi C & Puccetti P. Reverse signaling through GITR ligand enables dexamethasone to activate IDO in allergy. Nat Med 2007; 13:579-586; Nakamura T, Niimi S, Nawa K, Noda C, Ichihara A, Takagi Y, Anai M & Sakaki Y. Multihormonal regulation of transcription of the tryptophan 2,3-dioxygenase gene in primary cultures of adult rat hepatocytes with special reference to the presence of a transcriptional protein mediating the action of glucocorticoids. J Biol Chem 1987; 262:727-733). Therefore, there is a hypothetical link between lower levels of liver HBV DNA in female mice, and hormone-regulated tryptophan metabolism. Moreover, the trend for female HBV transgenic mice to have higher baseline levels of anti-HBV cytokines (Cavanaugh V J, Guidotti L G & Chisari F V. Interleukin-12 inhibits hepatitis B virus replication in transgenic mice. J Virol 1997; 71:3236-3243), such as IL-12 (Table 9), also implicates a role for the Th1 immuno-regulatory end products of IDO in HBV DNA inhibition.

Without wishing to be bound to any theory, the possible involvement of IDO in determining the levels of liver HBV DNA may explain the mechanism of anti-HBV DCP activity, since DCP is known to inhibit IDO in human PBMCs (Moheno P, Pfleiderer W, Dipasquale A G, Rheingold A L & Fuchs D. Cytokine and IDO metabolite changes effected by calcium pterin during inhibition of MDA-MB-231 xenograph tumors in nude mice. Int J Pharm 2008; 355:238-248) and upregulate the anti-HBV cytokine, IL-12, in nude mice (Moheno P, Pileiderer W & Fuchs D. Plasma Cytokine Concentration Changes Induced by the Antitumor Agents Dipterinyl Calcium Pentahydrate (DCP) and Related Calcium Pterins. Immunobiology in press). The higher IDO activity found in the sera of the female HBV mice might allow for a relatively greater degree of IDO inhibition by DCP, and consequently greater immuno-enhancement in these mice. FIGS. 18A, 18B, and 18C depict Trp, Kyn, and Kyn/Trp serum levels rather than tissue (hepatic) level where the therapeutic IDO inhibition is likely occurring. Higher hepatic IDO levels in the female HBV mice might serve as enhanced metabolic targets for DCP, and thereby sustain Th1 immune responses.

The generally higher serum Trp levels in the HBV females, versus the males, which disappear at 23.0 mg/(kg d) DCP, as well as the significantly higher Trp levels versus controls for the males at this dosage, indicate the likely involvement of IDO-related inhibition by DCP in the males (FIG. 18A). However, the serum Kyn/Trp does not change appreciably for the males (or females) with DCP (FIG. 18C), as serum Kyn levels may be maintained through metabolic mass action from serum Trp. The generally elevated serum levels of Trp, Kyn, and Kyn/Trp in the HBV females may reflect both a mass action effect from higher Trp with the females, as well as IDO-induction by estrogen. Higher serum Trp levels in the female HBV mice might also play a more direct anti-HBV role by boosting cytokines and immunity, generally. Here again, these IDO-related measures are distant to the metabolic situation at the hepatic tissue level.

The serum IDO differences between humans and mice might also relate to NOS differences because 1) human macrophages are deficient in high output NO production (Vazquez-Torres A, Stevanin T, Jones-Carson J, Castor M, Read R C & Fang F C. Analysis of nitric oxide-dependent antimicrobial actions in macrophages and mice. Methods Enzymol 2008; 437:521-538; Weinberg J B. Nitric oxide production and nitric oxide synthase type 2 expression by human mononuclear phagocytes: a review. Mol Med 1998; 4:557-591.), and 2) NO interferes with IDO expression and function (Suh H S, Mao M L, Rivieccio M, Choi S, Connolly E, Zhao Y, Takikawa O, Brosnan C F & Lee S C. Astrocyte indoleamine 2,3-dioxygenase is induced by the TLR3 ligand poly(I:C): mechanism of induction and role in antiviral response. J Virol 2007; 81:9838-9850). Thus, the lower serum Kyn/Trp (i.e., lower IDO activity) in mice as compared with humans, noted above, could relate to the higher NO levels in mice. Examination of the various possible factors influencing serum IDO is potentially of significant interest if IDO levels are found to play a significant role in the antiviral activity of immunotherapeutics such as DCP.

Several published findings regarding RANTES, a chemokine that promotes T cell activation and proliferation, and hepatitis B are worth noting. First, no significant change was found in the levels of RANTES expression for female BALB/c mice injected with plasmid DNA vaccines encoding the hepatitis B virus (HBV) surface envelope antigens (Nam S H, Park J H, Kang J H, Kang S Y, Kim J H, Kim S Y, Alm J I, Park K S & Chung H J. Modulation of immune response induced by co-administration of DNA vaccine encoding HBV surface antigen and HCV envelope antigen in BALB/c mice. Arch Pharm Res 2006; 29:1042-1048). Second, plasmid-encoded RANTES was found to polarize immune responses towards Th1 in female BALB/c mice co-infected with HBsAg plasmid, a response thought to be a prerequisite to HBV clearance (Ma K, Xu W, Shao X, Yanyue, Hu L, Xu H, Yuan Z, Zheng X & Xiong S. Communization with RANTES plasmid polarized Th1 immune response against hepatitis B virus envelope via recruitment of dendritic cells. Antiviral Res 2007; 76:140-149). Third, human case studies of RANTES polymorphisms determined that alone they are not associated with HBV recovery (Alm S H, Kim do Y, Chang H Y, Hong S P, Shin J S, Kim Y S, Kim H, Kim J K, Paik Y H, Lee K S, Chon C Y, Moon Y M & Han K H. Association of genetic variations in CCR5 and its ligand, RANTES with clearance of hepatitis B virus in Korea. J Med Virol 2006; 78:1564-1571; Cheong J Y, Cho S W, Choi J Y, Lee J A, Kim M H, Lee J E, Hahm K B & Kim J H. RANTES, MCP-1, CCR2, CCR5, CXCR1 and CXCR4 gene polymorphisms are not associated with the outcome of hepatitis B virus infection: results from a large scale single ethnic population. J Korean Med Sci 2007; 22:529-535; Thio C L, Astemborski J, Thomas R, Mosbruger T, Witt M D, Goedert J J, Hoots K, Winkler C, Thomas D L & Carrington M. Interaction between RANTES promoter variant and CCR5Delta32 favors recovery from hepatitis B. J Immunol 2008; 181:7944-7947). These findings, taken together with the significant serum RANTES decrease in the male HBV mice with 2.3 mg/(kg day) DCP (FIG. 17), imply a possible Th1/Th2 shift towards Th2 by DCP in the males at this dosage. However, because significant decreases in serum RANTES is only seen at this one DCP dosage, changes in RANTES serum levels are not likely strongly coupled to the inhibition of HBV replication.

A finding from this study is that DCP significantly inhibits hepatitis B virus replication, as measured by PCR, in female HBV transgenic mice in a dose-response manner (FIG. 19). DCP inhibition of tissue level IDO, to a greater extent than systemically dispersed serum IDO, might play a role in the anti-HBV effect by therapeutically reducing hepatic tissue kynurenine levels, and by increasing tryptophan availability, both of which enhance T cell-mediated immunity (Zamanakou M, Germenis A E & Karanikas V. Tumor immune escape mediated by indoleamine 2,3-dioxygenase. Immunol Lett 2007; 111:69-75; Temess P, Bauer T M, Rose L, Duller C, Watzlik A, Simon H & Opelz G. Inhibition of allogeneic T cell proliferation by indoleamine 2,3-dioxygenase-expressing dendritic cells: mediation of suppression by tryptophan metabolites. J Exp Med 2002; 196:447-457; Weinlich G, Murr C, Richardsen L, Winlder C & Fuchs D. Decreased serum tryptophan concentration predicts poor prognosis in malignant melanoma patients. Dermatology 2007; 214:8-14; Brandacher G, Cakar F, Winkler C, Schneeberger S, Obrist P, Bosmuller C, Wemer-Felmayer G, Werner BR, Bonatti H, Margreiter R & Fuchs D. Non-invasive monitoring of kidney allograft rejection through IDO metabolism evaluation. Kidney Int 2007; 71:60-67; Frumento G, Rotondo R, Tonetti M, Damonte G, Benatti U & Ferrara G B. Tryptophan-derived catabolites are responsible for inhibition of T and natural killer cell proliferation induced by indoleamine 2,3-dioxygenase. J Exp Med 2002; 196:459-468). Studies linking the collapse of HBV-specific CD8 T-cells, and impaired innate immunity (NK cells), in persons with chronic hepatitis B (CHB) have been reviewed (Wang F S. Clinical immune characterization of hepatitis B virus infection and implications for immune intervention: Progress and challenges. Hepatal Res 2007; 37 Suppl 3:S339-346). Reversal of these immunological debilities by DCP might be expected based upon its IDO-inhibitory activity.

TABLE 9
Additional Gender Effects^{‡}

| Viral or serum | | | | | |
|---|---|---|---|---|---|
| cytokine measure by | Female levels | | Male levels | | |
| treatment group | (mean ± SEM) | | (mean ± SEM) | | Significance |
| Liver HBV DNA (Southern) | | | | | |
| Adefovir dipivoxil | 3.5 ± 0.55 | pg/ug | 1.5 ± 0.37 | pg/ug | .038 |
| 2.3 mg/(kg d) DCP | 12.7 ± 1.9 | pg/ug | 46.4 ± 10.8 | pg/ug | .011 |
| 7.3 mg/(kg d) DCP | 17.3 ± 3.5 | pg/ug | 50.4 ± 10.3 | pg/ug | .007 |
| 23.0 mg/(kg d) DCP | 10.6 ± 1.6 | pg/ug | 36.0 ± 10.6 | pg/ug | .013 |
| HBcAg Stained nuclei/total | | | | | |
| 2.3 mg/(kg d) DCP | 0.36 ± 0.046 | | 0.51 ± 0.041 | | .026 |
| Adefovir dipivoxil | 27.7 ± 3.0 | | 14.1 ± 3.5 | | .016 |
| IL-4 | | | | | |
| 7.3 mg/(kg d) DCP | 241 ± 53 | rel. pg/ml | 87 ± 28 | rel. pg/ml | .019 |
| IL-9 | | | | | |
| 7.3 mg/(kg d) DCP | 185 ± 28 | rel. pg/ml | 112 ± 13 | rel. pg/ml | .030 |
| IL-12 | | | | | |
| 7.3 mg/(kg d) DCP | 642 ± 11 | rel. pg/ml | 575 ± 17 | rel. pg/ml | .009 |

pg/ug = picogram per microgram
rel. pg/ml = relative picogram per milliliter
^{‡}Other significant gender effects were found that might relate to the FIG. 5 DCP gender difference in DCP anti-HBV efficacy. Gender differences were found for all treatment groups on liver HBV DNA (Southern) and at single DCP dosages for certain serum cytokines. The positive control, adefovir dipivoxil, was found to induce gender differences on liver HBV DNA (Southern) and on the number of HBcAg stained nuclei per quarter field, as well.

### REFERENCES

Andondonskaja-Renz, B., Zeitler, H. J., 1983. Separation of pteridines from blood cells and plasma by reverse-phase high-performance liquid chromatography. Anal Biochem., 133, 68-78.
Andondonskaja-Renz, B., Zeitler, H. J., 1984. Pteridines in plasma and in cells of peripheral blood from tumor patients. In: Pfleiderer, W., Wachter, H. Curtius, H. Ch. (Eds.), Biochemical and Clinical Aspects of Pteridines: Cancer, immunology, metabolic diseases: Proceedings, Third Winter Workshop on Pteridines, Feb. 18-25, 1984, St. Christoph, Arlberg, Austria, Vol. 3, Walter de Gruyter & Co., Berlin; N.Y., pp. 295-311.
Blair, J. A., Pearson, A. J., 1974. Kinetics and mechanism of the autoxidation of the 2-amino-4-hydroxy-5,6,7,8-tetrahydropteridines. J. Chem. Soc. Perkin II, 80-88.
Eto, I., Bandy, M. D., Butterworth, C. E. Jr., 1992. Plasma and urinary levels of biopterin, neopterin, and related pterins and plasma levels of folate in infantile autism. J. Autism. Dev. Disord., 22, 295-308.
Johlin, F. C., Fortman, C. S., Nghiem, D. D., Tephly, T. R., 1987. Studies on the role of folic acid and folate-dependent enzymes in human methanol poisoning. Mol. Pharmacol., 31, 557-561.
Jones, S. A., 2005. Directing transition from innate to acquired immunity: defining a role for IL-6. J. Immunol., 175, 3463-3468.
Mocellin, S., Marincola, F. M., Young, H. A., 2005. Interleukin-10 and the immune response against cancer: a counterpoint. J. Leukoc. Biol., 78, 1043-1051.
Moheno, P., 2004. Calcium pterin as an antitumor agent. Int. J. Pharm., 271, 293-300.
Moheno P, Pfleiderer W, DiPasquale A, Rheingold A, Fuchs D. Cytokine and IDO Metabolite Changes Effected by Calcium Pterin During Inhibition of MDA-MB-231 Xenograph Tumors in Nude Mice. International Journal of Pharmaceutics, in press.
Moheno, P., Winkler, C., Fuchs, D., Ryan, J., Meerbergen, E., 2005. Role of calcium pterin in natural killer (NK) cell activation and indoleamine 2,3-dioxygenase (IDO) modulation for antitumor activity. Pteridines, 16, 140.
Munn, D. H., 2006. Indoleamine 2,3-dioxygenase, tumor-induced tolerance and counter-regulation. Curr. Opin. Immunol., 18, 220-225.
Schrocksnadel, K., Wirleitner, B., Winter, C., Fuchs, D., 2006. Monitoring tryptophan metabolism in chronic immune activation. Clin. Chim. Acta, 364, 82-90.
Uyttenhove, C., Pilotte, L., Theate, I., Stroobant, V., Colau, D., Parmentier, N., Boon, T., Van den Eynde, B. J., 2003. Evidence for a tumoral immune resistance mechanism based on tryptophan degradation by indoleamine 2,3-dioxygenase. Nat. Med., 9, 1269-1274.
Vicari, A. P., Trinchieri, G., 2004. Interleukin-10 in viral diseases and cancer: exiting the labyrinth? Immunol. Rev., 202, 223-236.
Weiss J M, Subleski J J, Wigginton J M, Wiltrout R H. Immunotherapy of cancer by IL-12-based cytokine combinations. Expert Opin Biol Ther. 2007 November; 7(11):1705-1721.
Widmer, B., Werner, E. R., Schennach, H., Wachter, H., Fuchs, D., 1997. Simultaneous measurement of serum tryptophan and kynurenine by HPLC. Clin. Chem., 43, 2424-2426.
Winkler, C., Schroecksnadel, K., Moheno, P., Meerbergen, E., Schennach, H., Fuchs, D., 2006. Calcium-pterin suppresses mitogen-induced tryptophan degradation and neopterin production in peripheral blood mononuclear cells. Immunobiolog., 211, 779-784.
Wirleitner, B., Neurauter, G., Schroecksnadel, K., Frick, B., Fuchs, D., 2003. Interferon-□-induced conversion of tryptophan: immunologic and neuropsychiatric aspects. Curr. Med. Chem., 10, 1581-1591.
Zeitler, H. J., Andondonskaja-Renz, B., 1987. Evaluation of pteridines in patients with different tumors. Cancer Detect. Prev., 10, 71-79.
Zeitler, H. J., Andondonskaja-Renz, B., Fink, M., Wilmanns, W., 1983. Pteridines in blood cells and plasma: Methods for the quantification of free and conjugated (protein-bound) pteridines. In: Curtius, H. Ch., Pfleiderer, W., Wachter, H. (Eds.), Biochemical and Clinical Aspects of Pteridines: Cancer, immunology, metabolic diseases: Proceedings, Second Winter Workshop on Pteridines, Mar. 6-9, 1983, St. Christoph, Arlberg, Austria, Vol. 2, Walter de Gruyter & Co., Berlin; N.Y., pp. 89-104.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A method of treating cancer comprising administration of a composition comprising calcium pterin.

2. The method of claim 1 wherein the calcium pterin has a stoichiometry of 1:4/calcium:pterin.

3. The method of claim 1 wherein the calcium pterin has a stoichiometry of 1:2/calcium:pterin.

4. The method of claim 1 wherein the calcium pterin is dipterinyl calcium pentahydrate (DCP).

5. The method of claim 1 wherein administration of the composition results in decreased IL-6 levels.

6. The method of claim 1 wherein administration of the composition results in increased IL-10 levels.

7. The method of claim 1 wherein administration of the composition results in decreased IFN-γ levels.

8. The method of claim 1 wherein administration of the composition results in increased kynurenine levels.

9. The method of claim 1 wherein administration of the composition results in increased IL-12 levels.

10. The method of claim 1 wherein administration of the composition results in decreased IL-6 levels.

11. The method of claim 1 wherein administration of the composition results in increased IL-4 levels.

12. The method of claim 1 wherein administration of the composition results in inhibition of indoleamine 2,3-dioxygenase.

13. The method of claim 1 wherein administering the composition is through oral, parenteral, intravenous, subcutaneous, intrathecal, intramuscular, buccal, intranasal, epidural, sublingual, pulmonary, local, rectal, or transdermal administration.

14. The method of claim 1 further comprising additional therapies selected from one or more of radiation therapy, chemotherapy, high dose chemotherapy with stem cell transplant, hormone therapy, and monoclonal antibody therapy.

15. The method of claim 1 wherein the cancer is selected from the group consisting of oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

16. A method of modulating the immune response comprising administration of a composition comprising calcium pterin.

17. A method of treating an inflammatory-based disease or disorder comprising administration of a composition comprising calcium pterin.

18. The method of claim 17 wherein the inflammatory-based disease or disorder is selected from infectious diseases, neurodegenerative disorders, multiple sclerosis, HIV-associate dementia, AIDS dementia, Alzheimer's disease, central nervous system inflammation, obesity, dementia (various forms), coronary heart disease, diabetes (Type 1 and Type 2), atherosclerosis, chronic inflammatory diseases, autism, neonatal onset multisystem inflammatory disease, (also known as NOMID, Chronic Neurologic Cutaneous and Articular Syndrome, or CINCA), Parkinson's Disease, rheumatoid arthritis, osteoarthritis, tendinitis, bursitis, inflammatory lung disease, psoriasis, chronic obstructive pulmonary disease, lupus erythematosus, organ inflammation (eg. myocarditis, asthma, nephritis, colitis), inflammatory bowel disease (IBD), autoimmune disease, inflammatory bowel syndrome (IBS), Crohn's Disease, Chronic Ulcerative Colitis, transplant rejection, sepsis, disseminated intravascular coagulation (DIC), septic shock, psoriasis, emphysema and ischemia-reperfusion injury.

19. The method of claim 17 wherein the inflammatory-based disease or disorder is hepatitis B virus infection.

20. The method of claim 17 wherein administering the composition is through oral, parenteral, intravenous, subcutaneous, intrathecal, intramuscular, buccal, intranasal, epidural, sublingual, pulmonary, local, rectal, or transdermal administration.

21. The method of claim 19 wherein administering the composition is through oral, parenteral, intravenous, subcutaneous, intrathecal, intramuscular, buccal, intranasal, epidural, sublingual, pulmonary, local, rectal, or transdermal administration.

22. The method of claim 19 further comprising additional therapies selected from one or more of interferon α, pegylated intereron α-2a, lamivudine, adefovir, tenofovir, telbivudine and entecavir.

23. The method of claim 19 wherein the calcium pterin is dipterinyl calcium pentahydrate (DCP).
